(19) United States
(12) Patent Application Publication
(10) Pub. No.: US 2024/0347184 A1
(43) Pub. Date: Oct. 17, 2024

(54) SYSTEMS AND METHODS FOR MANAGING PATIENT CARE

(71) Applicant: Pearl Health, Inc., New York, NY (US)

(72) Inventors: Alana Levy, New York, NY (US); Arlen James Young, Orlando, FL (US); Jennifer Rabiner, Brookline, MA (US); Lana Cohen, Arlington, MA (US); Wing Lun Ting, Needham, MA (US); Nellie Ponarul, Minneapolis, MN (US); Patrick Mauro, Seattle, WA (US); Peter Jamieson, Brookline, MA (US)

(73) Assignee: Pearl Health, Inc., New York, NY (US)

(21) Appl. No.: 18/633,845

(22) Filed: Apr. 12, 2024

Related U.S. Application Data
(60) Provisional application No. 63/458,746, filed on Apr. 12, 2023.

(51) Int. Cl. G16H 40/20; G16H 10/60

(52) U.S. Cl. CPC G16H 40/20; G16H 10/60

(57) ABSTRACT

Provided are systems and methods for managing patient care. Managing patient care involves obtaining one or more electronic patient health datasets related to a plurality of patients; generating a plurality of patient profiles from the one or more electronic patient health datasets, where a first patient profile of the plurality of patient profiles is generated by: analyzing the identified data to identify data associated with a first patient; analyzing data associated with the first patient to determine data associated with patient features and determining a severity score using the identified data; analyzing the data associated with the first patient to identify data associated with medical events of the first patient and determining an urgency score using the identified data; and displaying ordered representations of profiles of the plurality of patient profiles, wherein the representations are ordered based at least in part on the severity score of the respective profile.

### CROSS-REFERENCE TO RELATED APPLICATIONS

This Application is a Non-Provisional of Provisional (35 USC 119 (c)) of U.S. Application Ser. No. 63/458,746, filed Apr. 12, 2023, entitled “SYSTEMS AND METHODS FOR MANAGING PATIENT CARE”, which is incorporated by reference herein in its entirety.

### BACKGROUND

Modern healthcare locations, such as hospitals, private practices, and specialist practices, often provide care to hundreds or thousands of individual patients. To track patient conditions and care effectively, healthcare locations may employ the use of a patient management system. Patient management systems may store information related to patients, including patient diagnoses, medications, recent medical events, and past procedures, among other patient information. Effective management and displaying of patient information is important for medical practitioners to provide optimal care to their patients, which in turn, will improve patient outcomes.

Patient management systems may receive patient health information from multiple sources. For example, a patient management system may receive patient health information generated from within the environment to which it is deployed. This same patient management system may additionally receive patient health information generated outside of the environment from which it is deployed. A patient management system may receive patient health information from one or more sources external from the environment to which a system is deployed. Such sources may include Electronic Health Records (EHR) vendors, Electronic Medical Records (EMR) vendors, Insurance Providers, or Health Information Exchanges (HIE). Patient management systems may be available within the computer network of a healthcare location, may be available to multiple healthcare locations, and/or may be available to multiple providers of a patient across multiple healthcare locations. Patient management systems may be provided in private cloud computing environments (e.g., cloud infrastructure operated for one organization), public cloud computing environments (e.g., cloud infrastructure made available for use by others, for example, over the Internet or any other network, e.g., via subscription, to multiple organizations), a hybrid cloud computing environment (a combination of publicly-accessible and private infrastructure) and/or using any other type of cloud computing environment.

### SUMMARY

In some embodiments as discussed herein, improved systems and methods are provided for managing patients and their care. In some implementations, new systems and interfaces are provided to physicians and other health care workers to permit a proactive, patient-centric view of a patient group under care. This patient-centric displaying of patient medical information allows medical professionals to more effectively prioritize patients for care, better track patient care and needs and better track patient outcomes across a healthcare system.

According to one aspect a method for facilitating medical care of patients is provided. The method comprises using one or more processors to perform: obtaining one or more datasets related to a plurality of patients, generating a plurality of patient profiles having a respective patient profile for each patient of the plurality of patients, each respective patent profile is generated by: extracting data related to the patient from the one or more datasets, determining a respective severity score for the patient profile based at least in part on the extracted data, and determining a respective urgency score for the patient profile based at least in part on the extracted data, and displaying ordered respective representations of the plurality of patient profiles, wherein the respective representations are ordered based at least in part on the severity score of the respective profile.

According to one embodiment, determining the respective severity score comprises analyzing the extracted data for data related to at least one of: an age of the respective patient, a gender of the respective patient, and chronic conditions of the respective patient.

According to one embodiment, determining the respective severity score comprises analyzing the extracted data for data related to at least one of: an income associated with the respective patient, a location associated with the respective patient, an education level associated with the respective patient, an employment status associated with the respective patient, a family condition associated with the respective patient, one or more behaviors associated with the respective patient, a measure of health care access associated with the respective patient, acute conditions of the respective patient, and comorbidities associated with the respective patient.

According to one embodiment, processing the extracted data to determine a respective urgency score comprises determining, from the extracted data, a plurality of events related to the respective patient.

According to one embodiment, the plurality of events includes events selected from: medical facility admission, medical facility discharge, medical facility transfer, and medical provider actions.

According to one embodiment, the method further includes determining from the plurality of events a plurality of signals related to the respective patient, wherein each signal of the plurality of signals is determined from at least one event of the plurality of events.

According to one embodiment, at least one signal of the plurality of signals is determined using machine learning.

According to one embodiment, the plurality of signals includes signals selected from: time since admission to a medical facility, time since discharge from a medical facility, time until a recommended follow-up, time since a recommended follow-up, and time since last annual wellness visit.

According to one embodiment, the plurality of signals further includes signals selected from: a measure of prescription adherence, a probability of readmission, a measure of a need for advanced care planning and a measure of a need for specialist referral.

According to one embodiment, determining the respective urgency score comprises processing the plurality of signals and the severity score.

According to one embodiment, the method further comprising for each patient profile determining a suggested action plan, comprising a plurality of actions, based at least on the plurality of signals associated with the respective patient.

According to one embodiment, determining an action plan comprises analyzing each of the plurality of signals associated with the respective patient and adding at least one action associated with each signal of the plurality of signals to the plurality of actions of the action plan.

According to one embodiment, determining an action plan comprises analyzing each of the plurality of signals associated with the respective patient and adding at least one action associated with two or more signals of the plurality of signals to the plurality of actions of the action plan.

According to one embodiment, wherein the plurality of actions of the action plan comprises actions selected from: contact the respective patient via phone, contact the respective patient in medical facility, schedule an appointment for the respective patient, schedule an annual wellness visit for the respective patient.

According to one embodiment, the plurality of actions of the action plan further comprises actions selected from: refer the respective patient to a specialist, recommend a medical intervention for the respective patient.

According to one embodiment, an urgency score associated with a patient profile increases in response to an increase in the severity score associated with the patient profile.

According to one embodiment, an urgency score of a patient profile increases in response to time until a recommended follow-up crossing a threshold amount of time.

According to one embodiment, an urgency score of a patient profile increases in response to an increase in the time since admission signal, an increase in the time since discharge, and an increase in the time since transfer, until a signal indicating a follow-up step has been recommended for consideration has occurred or been considered is received.

According to one embodiment, the method further comprising determining one or more performance metrics based at least in part on changes in urgency scores associated with one or more patient profiles, changes in value of the time until a recommended follow-up signal, changes in value of the time since a recommended follow-up signal, and changes in value of the time since last annual wellness visit signal.

According to one embodiment, wherein an increase in the urgency score of a patient profile indicates the respective patient may receive particular benefit from proactive attention from medical providers.

According to one embodiment, wherein an increase in the severity score of a patient profile indicates the respective patient has experienced a decrease in overall health.

According to one embodiment, the method further comprising receiving data related to one or more events related to a particular patient of the plurality of patients; and in response to receiving data related to one or more events related to the particular patient, updating the respective urgency score for the respective patient profile.

According to one aspect a method for facilitating medical care recommendations for patients for provider consideration is provided. The method comprises obtaining one or more datasets related to a plurality of patients, processing the one or more datasets to determine a plurality of patient profiles, each patient profile of the plurality or patient profiles related to a respective patient of the plurality of patients, displaying, on a device screen, a patient map, wherein the patient map includes a graphical representation of each respective patient profile of a first subset of patient profiles of the plurality of patient profiles, wherein the patient map displays, for each graphical representation of each respective patient profile, an indication of urgency for attention for the respective patient.

According to one embodiment, the act of displaying the graphical representation of the plurality of patient profiles is displayed in an order on the device screen responsive to a last touchpoint with each of the respective patients.

According to one embodiment, the patient map is a rectangular map having a vertical axis and a horizontal axis, and wherein the graphical representations are ordered in a direction parallel to the vertical axis based on a severity score of the respective patient profile and the graphical representations are ordered in a direction parallel to the horizontal axis based on a signal indicative of days since last touchpoint of the respective patient profile.

According to one embodiment, the method further comprising displaying on the device screen a patient list, wherein the patient list includes a graphical list representation of each patient profile of the plurality of patient profiles.

According to one embodiment, the graphical list representations are ordered based on an urgency score of each respective patient profile.

According to one embodiment, the method further comprising displaying one or more patient map filters on the device screen.

According to one embodiment, the one or more patient map filters include a provider filter and a reason for urgency filter.

According to one embodiment, the method further comprising in response to receiving a user input to the provider filter, displaying a graphical representation of each respective patient profile of a second subset of patient profiles of the plurality of patient profiles, wherein each patient profile of the second subset of patient profiles is associated with a provider input by the user to the provider filter.

According to one embodiment, the graphical representation of each patient profile of the plurality of patient profiles has a color associated with an urgency score of the respective patient profile.

According to one embodiment, the graphical representation of each patient profile of the plurality of patient profiles has a size associated with an urgency score of the respective patient profile.

According to one embodiment, the graphical representation of each patient profile of the plurality of patient profiles has a shape associated with an urgency score of the respective patient profile.

According to one embodiment, in response to receiving a user selection of a particular graphical representation of a patient profile of the patient map, displaying a patient summary.

According to one embodiment, the patient summary includes patient demographic information, patient diagnoses, and patient events.

According to one embodiment, the patient summary includes an actions menu comprising one or more suggested actions for provider consideration associated with an action plan of the respective patient.

According to one embodiment, the method further comprising in response to receiving a user input to the actions menu, updating one or more actions of the action plan of the respective patient profile.

According to one embodiment, the method further comprises in response to receiving a user selection of a particular graphical representation of a patient profile of the patient map, displaying a detailed patient view on the device screen.

According to one embodiment, the detailed patient view comprises patient demographic information, patient activity information, patient diagnosis information, a patient summary, and patient medication information.

According to one embodiment, the detailed patient view comprises a log action button.

According to one embodiment, the method further comprises in response to a user input to the log action button, displaying an action menu associated with the respective patient profile.

According to one embodiment, the method further comprises displaying a plurality of tabs on the device screen.

According to one embodiment, the plurality of tabs includes a performance tab, and a patients tab.

According to one embodiment, the method further comprises, in response to a user selection of the performance tab, displaying a performance view on the device screen.

According to one embodiment, the method further comprising, further comprising calculating one or more performance metrics based on information contained within the patient profiles of the plurality of patient profiles and displaying the one or more performance metrics.

According to one aspect, a system for facilitating medical care recommendations for patients for provider consideration is provided. The system comprises at least one computer hardware processor and at least one non-transitory computer-readable storage medium storing processor-executable instructions that, when executed by the at least one computer hardware processor, cause the at least one computer hardware processor to perform a method for facilitating medical care of patients. The method comprises using one or more processors to perform: obtaining one or more datasets related to a plurality of patients, generating a plurality of patient profiles having a respective patient profile for each patient of the plurality of patients, each respective patent profile is generated by: extracting data related to the patient from the one or more datasets, determining a respective severity score for the patient profile based at least in part on the extracted data, and determining a respective urgency score for the patient profile based at least in part on the extracted data, and displaying ordered respective representations of the plurality of patient profiles, wherein the respective representations are ordered based at least in part on the severity score of the respective profile.

According to one aspect, at least one non-transitory computer-readable storage medium storing processor-executable instructions that, when executed by at least one computer hardware processor, cause the at least one computer hardware processor to perform a method for facilitating medical care of patients is provided. The method comprises using one or more processors to perform: obtaining one or more datasets related to a plurality of patients, generating a plurality of patient profiles having a respective patient profile for each patient of the plurality of patients, each respective patent profile is generated by: extracting data related to the patient from the one or more datasets, determining a respective severity score for the patient profile based at least in part on the extracted data, and determining a respective urgency score for the patient profile based at least in part on the extracted data, and displaying ordered respective representations of the plurality of patient profiles, wherein the respective representations are ordered based at least in part on the severity score of the respective profile.

According to one aspect, a system for facilitating medical care of patients is provided. The system comprises at least one computer hardware processor and at least one non-transitory computer-readable storage medium storing processor-executable instructions that, when executed by the at least one computer hardware processor, cause the at least one computer hardware processor to perform a method for facilitating medical care of patients. The method comprises obtaining one or more datasets related to a plurality of patients, processing the one or more datasets to determine a plurality of patient profiles, each patient profile of the plurality or patient profiles related to a respective patient of the plurality of patients, displaying, on a device screen, a patient map, wherein the patient map includes a graphical representation of each respective patient profile of a first subset of patient profiles of the plurality of patient profiles, wherein the patient map displays, for each graphical representation of each respective patient profile, an indication of urgency for attention for the respective patient.

According to one aspect, at least one non-transitory computer-readable storage medium storing processor-executable instructions that, when executed by at least one computer hardware processor, cause the at least one computer hardware processor to perform a method for facilitating medical care of patients is provided. The method comprises obtaining one or more datasets related to a plurality of patients, processing the one or more datasets to determine a plurality of patient profiles, each patient profile of the plurality or patient profiles related to a respective patient of the plurality of patients, displaying, on a device screen, a patient map, wherein the patient map includes a graphical representation of each respective patient profile of a first subset of patient profiles of the plurality of patient profiles, wherein the patient map displays, for each graphical representation of each respective patient profile, an indication of urgency for attention for the respective patient.

### BRIEF DESCRIPTION OF FIGURES

Various aspects and embodiments will be described with reference to the following figures. It should be appreciated that the figures are not necessarily drawn to scale. Items appearing in multiple figures are indicated by the same or a similar reference number in all the figures in which they appear.

FIG. 1 illustrates an example environment and system in which aspects of the present invention may be deployed, in accordance with some embodiments of the technology described herein;

FIG. 2 is a diagram illustrating a process flow for determining portions the data to be included in a patient profile from obtained patient health information, in accordance with some embodiments of the technology described herein;

FIG. 3 illustrates an exemplary diagram of the severity scoring section of an exemplary system, in accordance with some embodiments of the technology described herein;

FIG. 4A illustrates an exemplary diagram of an event and signal determination section of an exemplary system, in accordance with some embodiments of the technology described herein;

FIG. 4B illustrates an example machine learning model which may be implemented in a signal determination module which calculates signal values, in accordance with some embodiments of the technology described herein;

FIG. 5A illustrates an exemplary urgency scoring module according to embodiments of the present invention, in accordance with some embodiments of the technology described herein;

FIG. 5B illustrates an example of a patient's urgency score changing over time in response to different events and actions, in accordance with some embodiments of the technology described herein;

FIG. 6 illustrates an example of an action plan section of an exemplary system, in accordance with some embodiments of the technology described herein;

FIG. 7 illustrates a representation of a patient profile and information which may be stored by an exemplary system, within a patient profile, in accordance with some embodiments of the technology described herein;

FIG. 8A illustrates a process for preparing patient profiles and displaying representations of patient profiles, in accordance with some embodiments of the technology described herein;

FIG. 8B illustrates a process which may be performed by an exemplary system to generate a patient profile related to a respective patient, in accordance with some embodiments of the technology described herein;

FIG. 8C illustrates a process which may be performed by a system to generate and display patient profiles, in accordance with some embodiments of the technology described herein;

FIG. 8D illustrates a process 830 which may be performed by a system for displaying patient profiles, in accordance with some embodiments of the technology described herein;

FIG. 9 illustrates an example process which may be followed during the implementation and use of an exemplary system, in accordance with some embodiments of the technology described herein;

FIG. 10 depicts a data processing section of the system which may be used to calculate one or more healthcare performance metrics, in accordance with some embodiments of the technology described herein;

FIG. 11A illustrates an example of a graphical user interface (GUI) for displaying data related to multiple patient profiles within a healthcare network, in accordance with some embodiments of the technology described herein;

FIG. 11B illustrates an example of a GUI of an exemplary system for displaying a patient summary alongside a patient map, in accordance with some embodiments of the technology described herein;

FIG. 11C illustrates the actions which a provider may select from the patient summary view of a GUI, in accordance with some embodiments of the technology described herein;

FIG. 12 illustrates a detailed patient view which may be provided by a GUI, in accordance with some embodiments of the technology described herein;

FIG. 13 illustrates a view of a GUI when a performance tab is selected, in accordance with some embodiments of the technology described herein;

FIG. 14 shows a block diagram of an exemplary computing device, in accordance with some embodiments of the technology described herein, in accordance with some embodiments of the technology described herein.

### DETAILED DESCRIPTION

As discussed above, it is important to provide effective management of patient data for healthcare providers in order to administer optimal care to patients. Patient data management may involve organizing patient information based on patient conditions, activities and events which have occurred related to the patients.

The inventors have recognized that conventional techniques for patient data management could be improved upon. Conventional patient data management techniques may store and display patient health information to providers, who may analyze the information to determine which patients require action. Conventional techniques may allow a provider to view patient records manually using various different methods to determine what actions, if any, should be taken. Such conventional techniques of patient data management do not provide an indication to a provider of which patients would most benefit from proactive action and it is reliant on the providers to determine and decide which patients to take action on. This may result in some patients not receiving the care they require as providers are unable to discern the needs of these patients from conventional patient data management techniques.

Accordingly, the inventors have developed a new technique for patient data management, involving analyzing patient health information, calculating one or more scores for the patient, and providing the patient scores and information to healthcare providers. The technique may involve obtaining patient health information and processing this information to determine one or more events associated with the patient. The events associated with a patient may be used to determine one or more signals for the patient, which are used to calculate an urgency score for the patient, indicative of how important proactive provider action is likely to be for that particular patient. As a result of analyzing and scoring patients for provider action, and unlike conventional patient data management techniques, the techniques developed by the inventors substantially streamline the workflow for a healthcare provider, allowing for more efficient and effective care to be delivered to patients. Such information may be presented to healthcare providers in an efficient interface that permits quick location of patients who require care and/or check-ins on their status.

In some embodiments, patient health information may be analyzed. Patient health information may be stored using one or more data structures which are accessible by a computer.

Such patient health information may include data indicative of patient demographics, social determinants of health (SDOH) information related to a patient, patient medical conditions, medical events related to a patient, prescriptions of a patient, and diagnoses of a patient. Patient demographic information may include one or more of: an age of a patient, and a gender of the patient, among other information. SDOH information may include one or more of: a patient income, a patient location, a patient education level, a patient employment status, a patient family condition, one or more patient behaviors, and a measure of patient health care access. Medical events related to a patient may include one or more of: medical facility admission, medical facility discharge, medical facility transfer, and medical provider actions such as medical procedures, tests and interventions. Prescriptions of a patient may include one or more of: prescriptions that are written for a patient, and the dates when prescriptions are collected by a patient.

In some embodiments, patient health information may be generated at medical locations by healthcare providers or medical professionals. Healthcare locations may include, but are not limited to, hospitals, private clinics, specialist clinics, urgent care clinics, pharmacies, and emergency departments, among other locations. Patient health information may be generated by healthcare providers or medical professionals including, but not limited to: doctors, nurses, physician assistants, and other members of a patient care team; emergency response professionals; lab technicians; and pharmacists, among other healthcare providers and medical professionals. Generating patient health information may include inputting relevant patient information to an electronic system interface, where the health information may be stored in one or more data structures. Patient health information may be prepared by healthcare providers and medical professionals by uploading relevant information to a patient's insurance provider, Electronic Medical Record or Electronic Health Record, which may then be accessed or obtained electronically. Patient health information may be stored in an electronic format, for example by using data structures which are computer readable and writable.

In some embodiments, patient health information is received from sources external to the network to which the system is deployed. Examples of such sources may include one or more of Electronic Health Records (EHR) vendors, Electronic Medical Records (EMR) vendors Insurance Providers, or Health Information Exchanges (HIE). Electronic Health Records (EHR) vendors, Electronic Medical Records (EMR) vendors and Health Information Exchanges (HIE) allow for the transmittal of patient health information between providers, clinicians and others within a patient's medical care network. EHR vendors, EMR vendors and HIEs may provide patient medical information in electronic formats including, but not limited to: tab-separated values (TSV), Health Level 7 (HL7), JavaScript Object Notation (JSON), and Fast Healthcare Interoperability Resource (FHIR). Patient health information may additionally be obtained from insurance providers as claims data. Claims data may be obtained from payors and may be structured in any suitable way, as discussed herein. Specific examples of external sources which may provide patient health information to a system may include Bamboo Health, Collective Health, and Experian Health.

In some examples, patient health information may be obtained for analysis via pull-based processes, in which a system requests patient health information from EMR vendors, EHR vendors, Insurance Providers or HIEs, which then provide the information to the system. Patient health information may also be obtained via push-based processes, in which EMR vendors, EHR vendors, Insurance Providers or HIEs send patient medical information to a system, without the system requesting the information. Push and pull based processes for the transmittal of patient health information may be achieved through application programming interface (API) calls to software provided by the vendors or by the system. Push and pull based processes for the transmittal of patient health information may occur based on a schedule, for example a system may query EMR vendors, EHR vendors, Insurance Providers or HIEs at set time intervals for patient health information or may occur in response to new information becoming available, for example EMR vendors, EHR vendors, Insurance Providers or HIEs may transmit patient health information to a system as new patient health information is received.

In some examples, a system may obtain patient health information for analysis via EHR integrations in the system. The system may request patient health information from the EHR integrations, and patient health information may be input into the system via an EHR user interface.

In some embodiments, obtained patient information may be analyzed to determine one or more events which have occurred related to a patient. The obtained patient information may include data related to the one or more events, including a type of event, a location of an event, a time of an event, an outcome of an event and one or more values associated with an event, among other data.

In some embodiments, the data about the one or more events may then be used to generate a value for one or more signals related to the patient. Examples of signals for a patient include but are not limited to: time since admission to a medical facility, time since discharge from a medical facility, time until a recommended follow-up, time since a recommended follow-up, time since last annual wellness visit, a measure of prescription adherence, a probability of readmission, a measure of a need for advanced care planning, a measure of a need for specialist referral and other signals as discussed herein. Values for such signals may be determined by a system according to a set of rules about events and data associated with events or may be determined based on a machine learning model. A machine learning model may be trained across groups of patients having common attributed and may output signals for patients in specific groups. A signal may be determined based on one or more events from the obtained patient information.

In some embodiments, a suggested action plan may be developed for a patient based on one or more signals associated with the patient. A suggested action plan may include one or more actions that a medical provider should consider for the associated patient. Examples of actions which may be provided for medical provider consideration include but are not limited to: contact a patient remotely, contact a patient in a medical facility, schedule an appointment for a patient, schedule an annual wellness visit for a patient, refer a patient to a specialist, and recommend a medical intervention for a patient. Actions may be suggested based on one signal associated with a patient or may be suggested by multiple signals associated with a patient. In addition, one signal may result in multiple actions being added to an action plan. Certain signals may result in specific actions being added to a patient's action plan. For example, a days since discharge signal of 0 may result in a schedule an appointment action being added to the action plan of the respective patient promptly following discharge.

In some examples, actions within an action plan may have one or more features. Features of actions may include one or more of: a type of action, a time of action, a reason for the action, an action category, notes on the action and an action status.

In some examples, an action category is an indication of the action's relation to patient care. Examples of action categories may include touchpoints and follow-ups. A touchpoint may be any action in which a patient is in direct contact with a medical provider, and/or medical staff. A follow-up may be an action where a patient is in direct contact with a medical provider and/or medical staff after an admission, discharge, or transfer type event.

In some examples, an action status is an indication of any activity a provider has taken to resolve a particular action. Examples of action statuses may include: New—the action is being seen for the first time or no activity has been taken to resolve the action, Hold—the action has been acknowledged by the user however no activity has been taken to directly resolve the action, Completed—the user has completed the action, Dismiss—the user has decided no activity is necessary to resolve the action. Completed actions may include scheduled a new appointment and checked in with patient and/or external provider. Hold actions may include awaiting next steps from the patient and/or external provider. Dismiss from queue actions may include already scheduled/contacted prior to urgency indication, unable to contact after multiple attempts, and alert is not relevant.

In some embodiments, a system may generate a profile related to a patient. A patient profile may store information related to a patient, such information may include, but is not limited to: events associated with a patient, signals associated with a patient, patient demographic information, a suggested action plan associated with a patient, patient prescriptions, one or more identifiers associated with a patient, one or more scores associated with a patient, and a summary associated with a patient. A patient profile may be maintained and updated by the system as additional information related to the patient is obtained. In addition, patient profiles may be accessed by a provider or medical professional to analyze a patient's status.

In some embodiments, a system may generate one or more scores related to a patient. For example, a system may generate a severity score for a patient, which provides an indication of the likelihood that a patient will benefit from proactive outreach. A severity score may be determined by information obtained about a patient, for example patient demographic information as discussed herein, SDOH data associated with a patient as described herein, chronic conditions of a patient, and comorbidities associated with a patient. A system may additionally generate an urgency score associated with a patient, which provides an indication of the current need for proactive provider action. An urgency score may be generated by processing signals and a severity score associated with a patient. A provider may utilize the severity and urgency scores of multiple patients to determine which patients require temporal prioritization over others. The scoring of patients ensures providers can effectively distribute care to patients, which in turn, improves the quality of healthcare and outcomes patients receive.

In some embodiments, a user, such as a medical professional may access a system which displays the patient health data, including information related to patient features, medical events related to patients, and urgency and severity scores of patients. In some examples, the health data for multiple patients may be displayed to the user of the system based on one or more of the severity scores of patients, and the urgency scores of patients. Displaying patient data organized based on the urgency score or the severity score determined for patients provides advantages over traditional patient data management systems because it provides a display of patients based on predicted patient needs, improved access to patient data, and improves access to patient care progression. This allows providers to better prioritize care for those patients who may most likely benefit from some medical intervention. This leads to improved workflow for providers and empowers providers to meet and address patient needs, even when the patient has reached out to the provider or otherwise indicated a need for medical attention—this can lead to improved patient outcomes and improved efficiency for medical providers and locations.

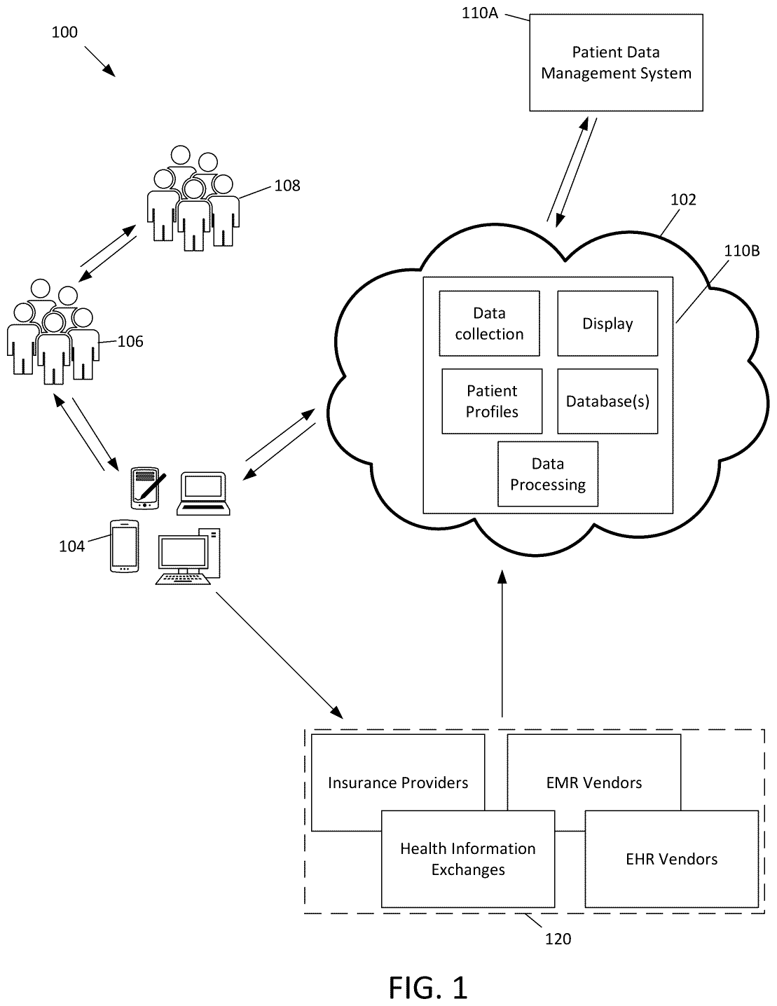

FIG. 1 illustrates an example environment 100 and system 110 in which aspects of the present invention may be deployed. The system 110 may be deployed within a healthcare location, multiple healthcare locations, or a healthcare network. One or more portions of the system may be implemented by a cloud computing network or similar computing network 102. These portions of the system 110B may function to store, distribute and process information to and from one or more external devices, for example devices where users may view, and provide patient health information. One or more portions of the system 110A may be implemented in computer hardware external to a cloud computing environment. These portions of the system may function to enable the storage, distribution and processing of the portions of the system implemented within the cloud computing environment. The system may include one or more computer hardware processors which perform the functions of the system.

In some examples, the system 110 may be accessed by users 106 within a healthcare location, at multiple healthcare locations, within a healthcare network or remotely. The system may be accessed using one or more devices 104, including, but not limited to, a desktop computer, a laptop computer, a mobile device, a smartphone, a tablet, a specialized terminal, among other devices. Multiple users 106 may access the system at a single location; therefore, it may be desirable for the system to provide features allowing providers to view information related to their specific patients, actions, and workflow.

In some examples, the system may include multiple sections such as a data processing section, patient profiles section, one or more databases, a data processing section, and a display section. These sections of the system represent different capabilities of the system and may not be implemented as physically separated or otherwise separated. The data processing section may process data from one or more of the databases, data collection section and the patient profiles section to determine one or more metrics associated with the network 102. The patient profile section may perform processes related to maintaining patient profiles within the system. The display section may perform processes related to the displaying of related information for one or more users. The databases may store information related to one or more patients, patient profiles, rules, and any other information necessary for the system to operate.

In some examples, the system may be accessed by one or more users 106, who may include any healthcare provider or any individual related to providing healthcare within an environment where the system is deployed. The one or more users may interact with both patients 108 and the system 110. Each user 106 may interact with one or more patients 108, and therefore may interact with one or more patient profiles within the system 110. When interacting with a patient 108, a user 106 may generate one or more pieces of patient health information. Such patient health information may include a patient diagnosis, provider actions related to the patient, measured patient data, new patient medications, patient notes, patient symptoms, patient events, imaging records, lab results and/or any other patient information as discussed herein. This generated patient health information may be inputted into the system directly by the user 106 for processing. This generated patient health information may additionally, or alternatively, be input into an EMR or EHR associated with the patient and received by system 110 from external systems 120.

In some examples, the system 110 may include one or more sections capable of communicating with one or more external systems 120, such as the data collection section. The one or more external systems may provide patient health information to the system. Examples of external systems may include Electronic Health Records (EHR) vendors, Electronic Medical Records (EMR) vendors, health insurance carriers, and/or Health Information Exchanges (HIE). Information may be obtained using push-based methods, pull-based methods or a combination of push and pull-based methods as discussed herein. It may be desirable for the system to communicate with external systems 120 to obtain patient health information which has been generated outside of the location, locations, or network to which the system 110 is deployed. The system may also communicate with external systems 120 to obtain patient health information which has been generated within the location, locations, or network to which the system 110 is deployed. External systems 120 may receive data generated within the location, locations or network to which the system is deployed via user 106 inputs to devices 104. Examples of such data may include data generated during a patient visit to an emergency room at a hospital external to any location where the system is deployed. Additional examples of such data include historical patient data generated at locations other than a location where the system is deployed. Collected patient health information may be used by the patient profiles section to generate respective patient profiles for each patient served by providers 106 connected to network 102.

In some embodiments, system 110 may facilitate transactions between outside systems 120 and system 110 or between system 110 and any other suitable system. System 110 may facilitate transactions by providing one or more of patient health information, information contained within patient profiles, and performance information to the outside systems. In some examples, the system 110 may provide a value-based care metric indicative of healthcare value generated by the deployment of system 110, to outside systems. The outside systems may initiate one or more processes in response to the facilitating information received from system 110. For example, in some embodiments, the value-based care metric may be used by an outside system (e.g. outside systems 120) to determine renumeration for a medical practice to which the system is deployed or for practitioner who uses the system based on certain performance metrics.

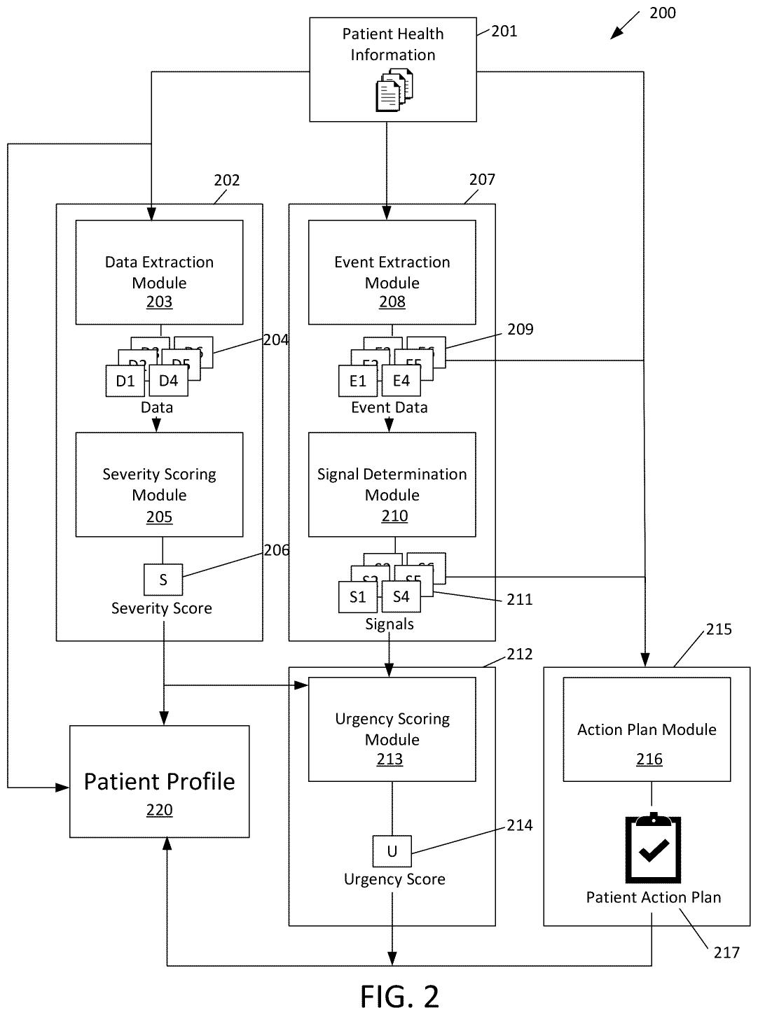

FIG. 2 is a diagram illustrating a process flow for determining portions of the data to be included in a patient profile from obtained patient health information. The process of FIG. 2 may be performed within a system, such as system 110 of FIG. 1. The patient profile section may receive patient health information 201 from a data collection section of the system. The patient health information 201 may have come from external sources, may have been generated by users or providers in the same network as the system, or may have come from sources both external and internal to the same network as the system, as discussed herein. The obtained patient health information 201 may be used and processed by a variety of modules and the outputs of these modules may contribute to the data within a patient profile.

The example of FIG. 2 illustrates a process flow for patient health information of a single patient contributing to the patient profile associated with the patient. The same process may be repeated for all patients treated by providers connected to the same network of the system, to generate a respective patient profile for each patient. The process of FIG. 2 may be performed for a subset of the patients treated by providers in the healthcare location(s) to which the system is deployed.

The process flow illustrated in FIG. 2, or any portion of the process flow, may be performed according to a schedule, wherein after a set period of time has passed, patient health information is analyzed, and the patient profile receives updated information, such as patient health information, patient scores or an action plan. In some instances, the patient profile may not change after scores are re-calculated or an action plan is re-determined. In other examples, the patient profile score may be updated when new patient health information is received. For example, if updated patient demographic information is received, the patient severity score may be re-calculated using the updated patient demographic information, and the patient profile may receive an updated severity score.

In the example of FIG. 2, patient health information 201 may be passed to a severity scoring section 202 which outputs a severity score 206 to be stored within the patient profile 220. The patient health information 201 includes electronic patient health information, as described herein, which may be stored using one or more data structures, and which may be analyzed by system 200. System 200 is described as having multiple sections and modules which perform different functions, however these sections and modules may not be separated and may be implemented to perform one or more functions. Within the severity scoring section 202, there is a data extraction module 203, which receives the patient health information 201 and extracts data 204 relevant to the patient severity scoring, from the one or more data structures used to store the patient health information, to be used in the severity scoring module 205. Data 204 that may be used for patient severity scoring is associated with one or more patient features and may include but is not limited to: patient demographic data as described herein, SDOH data associated with a patient as described herein, chronic conditions of a patient, and comorbidities associated with a patient. An additional example of data 204 which may be used in determining a patient's severity score is a Risk Adjustment Factor (RAF) or similar metric, which may be provided by Medicare or an insurance carrier of the patient. A RAF is a value associated with a patient, based on their age, gender and chronic conditions, which provides an indication of their need for proactive medical outreach. The previously mentioned data may be used as inputs to the severity scoring module 205.

In some examples, the severity scoring module 205 may receive data 204 from the data extraction module 203 and use this data 204 to calculate the severity score 206 for the patient associated with the data. The severity score 206 provides an indication of the overall health of the patient. In some examples, it is appreciated that a higher severity score may indicate that the patient has worse overall health and/or is more likely to have a greater need for proactive outreach and attention from a healthcare provider. The input data 204 may be processed in a variety of ways. For example, weights may be applied to the input data, and the weighted values are then used to determine the severity score. After being calculated, the patient severity score 206 may be sent to the patient profile 220 and to the urgency scoring module 213.

In some examples, the patient health information 201 may also be sent to an event and signal determination section 207 of the system. Within this section, the patient health information may be passed to an event extraction module 208. The event extraction module 208 may analyze the data contained within the patient health information 201 and associate certain data with one or more medical events related to the patient to generate event data 209. When extracting event data 209, the event extraction module 208 may associate one or more pieces of data with a particular event. For example, a type of event, time of event, and a location of the event, may be associated with a particular event. Examples of event types include, but are not limited to: medical facility admission, medical facility discharge, medical facility transfer, laboratory test results, and medical provider actions, among other events as discussed herein. In some examples, specific event types may receive additional information, such as a medical facility transfer event, may include information related to where a patient was located, for example, in an emergency department and where the patient was transferred to, for example, outpatient care.

In some examples, the event data 209 extracted by the event extraction module 208 may then be passed to the signal determination module 210. The signal determination module determines one or more signals 211 associated with a patient from the event data 209. A signal 211 may be a value associated with a certain aspect of a patient's medical care or condition. Signals may be based on a single event, or a plurality of events determined by the event extraction module 208. Examples of signals 211 include but are not limited to: time since admission to a medical facility, time since discharge from a medical facility, time until a recommended follow-up, time since a recommended follow-up, time since last annual wellness visit, a measure of prescription adherence, a probability of readmission, a measure of a need for advanced care planning, and a measure of a need for specialist referral, among other signals as discussed herein. Signals 211 may be determined in various ways.

For example, some signals 211 may be associated with values which can be directly measured based on event data 209. For example, some time-based signals may be determined by analyzing the time that has passed since an associated event. If a patient discharge event happened 3 days ago, the time since discharge from a medical facility signal value would be 3 days. In other examples, if event data is received that indicate a patient has refilled their prescription two out of the last four times, a signal indicating a measure of prescription adherence may be set to, for example, 2 refills/4 expected refills, 0.5, or 50%.

In other examples, a set of rules may be used to determine the values for certain signals of signals 211. These rules may associate specific event data 209, related to an event or a set of events with a specific value, for example, a rule for the signal indicating a measure of prescription adherence may state the signal value is increased by 1 every time a prescription is not refilled. If, for example, a patient did not refill their prescription two out of the last four times, the signal value would be 2.

In other examples, a machine learning model may be used to determine the value of particular signals of signals 211 associated with a patient. The machine learning model may be trained on historical patient data for a particular patient or on historical patient data for a group of patients within a healthcare network. For example, a probability of readmission signal value may be determined by a machine learning model. If, for example, a patient over the age of 65, having multiple chronic conditions, was admitted to the hospital for pneumonia, the machine learning model may be trained on historical data from a group of patients over the age of 65, with the same or similar chronic conditions to the patient. The model may then take as inputs, information about the patient and data about the admission event and output a signal indicating the probability of readmission of the patient.

In some examples, the signal values 211 may then be passed to an urgency scoring section 212 of the system. Within this section 212, the signal values 211 associated with the patient are processed in addition to the patient's severity score 206 and used to calculate an urgency score 214 for the patient. The urgency score 214 for the patient is an indication of a patient's current need for proactive attention from medical providers. In some embodiments, the urgency score 214 may be used by practitioners and medical professionals to prioritize patients for action and/or attention. In some embodiments, the urgency score provides an indication of a patient's need for proactive medical attention and/or action. The urgency score 214 may be determined by inputting the signal values 211 and the severity score 206 into a formula and calculating the urgency score using this formula. The patient urgency score formula may change over time to better account for user preferences. After calculation, the patient urgency score 214 is passed to the patient profile 220.

In some examples, the system may also include an action plan section 215, which may receive as inputs the patient health information 201, extracted event data 209 and the signal values 211 determined in the event and signal determination section 207. This information is used to determine a suggested action plan 217 associated with the patient. The action plan 217 may include one or more actions for a medical provider to consider with relation to the patient. Examples of actions include but are not limited to: contact a patient remotely, contact a patient in medical facility, schedule an appointment for a patient, schedule an annual wellness visit for a patient, refer a patient to a specialist, and recommend a medical intervention for a patient, among other actions as discussed herein. Actions may be determined in various ways.

For example, actions may be determined according to rules associated with received signals 211. An example of an action determined by a rule may be: every time a signal indicating a time since discharge is 0 days is received, a contact patient remotely action is added to the action plan. Each action may additionally have certain associated features, as discussed herein determined by one or more rules or inputs. For example, if the action plan module 216 receives data indicating a patient has been diagnosed with asthma, a refer patient to specialist action may be added to the action plan, and a specialist feature of the action may be a pulmonologist. The specialist feature would be determined by rules associating certain diagnoses with associated specialists. After determining an action plan 217 for a patient, the action plan stored patient profile 220, as described herein. In some examples, a user of the system may review the suggested actions in the action plan to determine their applicability to the patient, and the users may consider, modify and/or complete actions in the action plan.

In some examples, the patient profile may be stored using one or more data structures, as described herein. The patient profile may be stored in a data store of the system. In some examples, the system may include non-volatile storage, and/or may be connected to one or more cloud databases which may be used to store or maintain patient profiles and/or electronic patient health information.

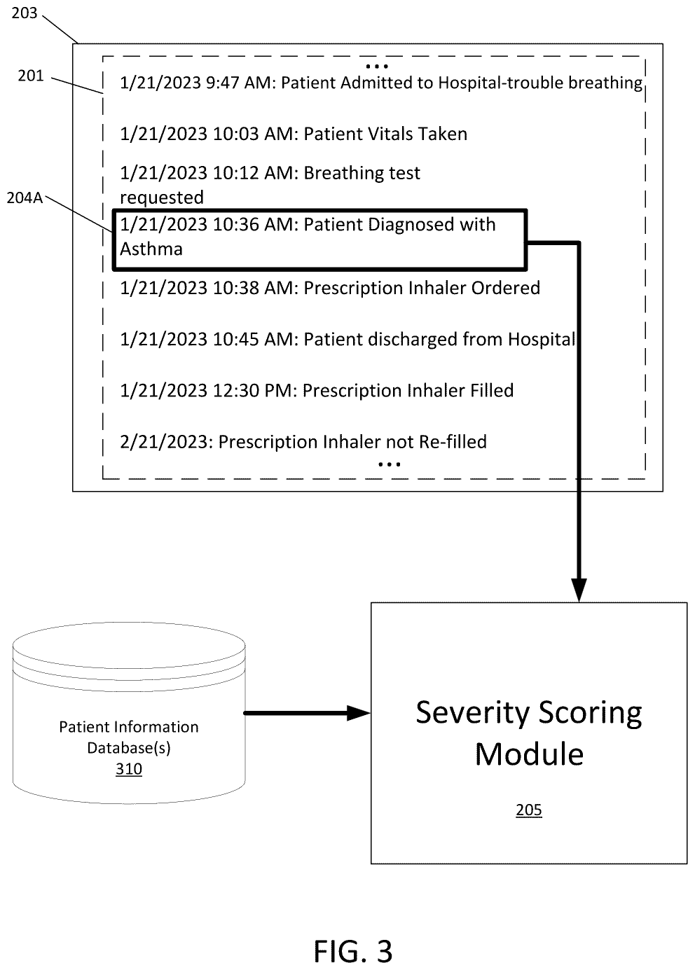

FIG. 3 illustrates an exemplary diagram of a severity scoring section of the exemplary system. The severity scoring section includes a data extraction module 203, which may analyze received patient health information 201 and extract data 204A relevant to the severity score calculation from the patient health information 201. In some examples, the severity scoring section may analyze a subset of patient health information 201. In the illustrated example, patient health information 201 includes information generated when a patient visited a hospital because of troubled breathing, was diagnosed with asthma at the hospital, and received a prescription for an inhaler. In this example, the information relevant to the severity score calculation is that the patient was newly diagnosed with asthma because this is a new chronic condition of the patient. This data 204A is identified by the data extraction module 203 and sent to the severity scoring module 205.

In some examples, the severity scoring module may additionally receive information from one or more databases 310 storing information related to patients. This information may include data necessary for calculating the severity score of a patient, for example data related to patient features, patient demographic data, patient SDOH data, and other patient diagnoses, among other data as described herein. The one or more databases 310 may be maintained by the system, for example as patient profiles, or may be external to the system such as external systems as discussed herein. The severity scoring module may request data from the one or more databases each time the severity score for a particular patient is calculated. The severity scoring module may request additional data from the one or more databases 310 because not all patient data required to calculate the severity score may be received in patient health information 201 received at a given time. Therefore, if data previously available or maintained by the system required for severity score calculation is not included in patient health information, the severity scoring module will request this data from the one or more databases.

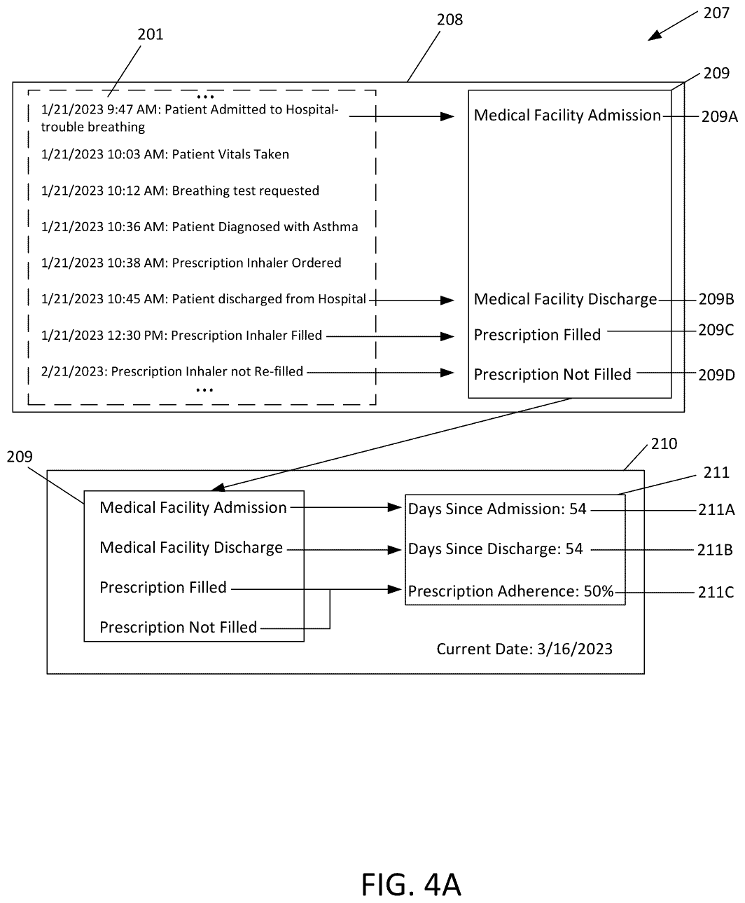

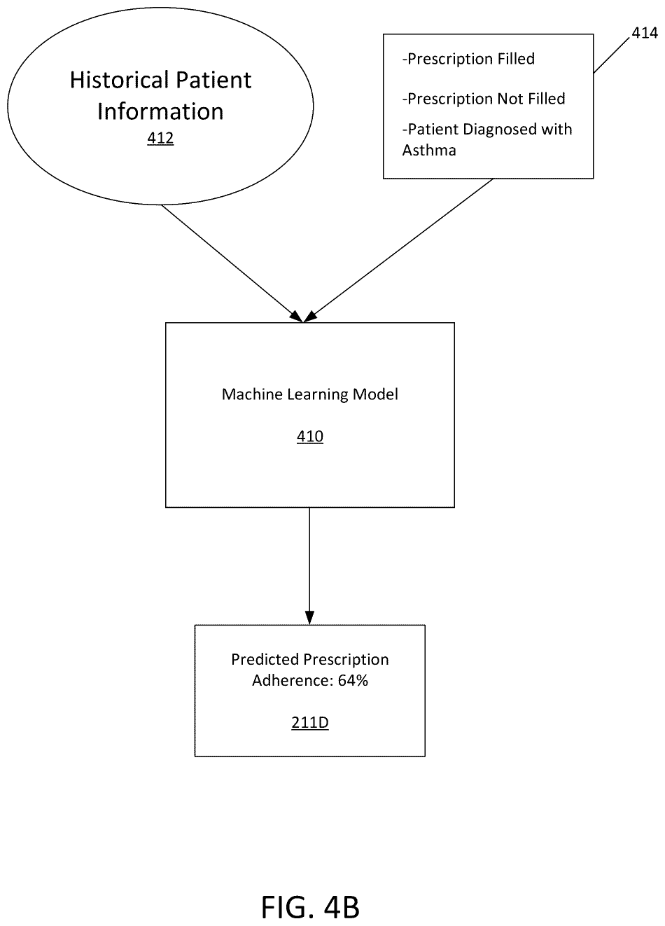

FIG. 4A illustrates an exemplary diagram of an event and signal determination section 207 of the system. The event and signal determination section 207 takes as inputs, patient health information 201 and outputs signals 211 related to the patient and event data 209 related to the patient. In some examples, the event and signal determination section may analyze a subset of patient health information 201. In the example of FIG. 4A, the same patient health information 201 as FIG. 3 is provided to the event and signal determination section 207. The patient health information 201 is passed to event extraction module 208, which analyzes the information and determines event data 209 related to one or more events related to the patient. In this example, the event extraction module may identify event data 209 related to admission to a medical facility, transfer to a medical facility, discharge from a medical facility, laboratory test result(s) received, and prescription fulfillment. The event extraction module identified event data 209 relevant to four events from the patient health information: a medical facility admission event, a medical facility discharge event, and two prescription-related events. Data associated with these identified events 209 may be passed to the signal determination module 210.

In some examples, within the signal determination module 210, event data 209 is analyzed to determine signal values 211 associated with the events. In some examples, signal values 211 may be determined by direct measurement. For example, the medical facility admission event 209A results in a days since admission signal 211A being generated based on the date of the medical facility admission and a current date determined by the system, in this case the signal value is 54 days. The medical facility discharge event 209B results in a days since discharge signal 211B being generated based on the date of the medical facility admission and a current date determined by the system, in this case the signal value is 54 days.

In some examples, signal values 211 may also be determined through rules or calculations. For example, the prescription adherence signal value 211C provides a measure of the patient's prescription adherence determined through calculation. The signal determination module determines the patient has filled their prescription one out of two possible times and therefore determines a prescription adherence value of 50% based on the received event data. The days since discharge signal 211B and days since admission signals 211A are each generated based on a single event, however some signals may be based on multiple events, for example the prescription adherence value 211C considers both the Prescription Filled event 209C and the Prescription Not Filled 209D events. In some examples, signal values may also be determined using other methods.

For example, FIG. 4B illustrates an example machine learning model 410 which may be implemented in the signal determination module 210 which determines one or more signal values. A signal determination module 210 may utilize any method to calculate signal values or may utilize a combination of methods, for example, one or more of rules, direct measurement, or machine learning, among other methods. The machine learning model 410 of FIG. 4B is configured to output a signal value related to patient prescription adherence. The machine learning model 410 may be trained on historical patient information 412 related to a group of patients. The group may contain hundreds, thousands, tens of thousands, hundreds of thousands or millions of patients. The model 410 may take as inputs information related to a patient, including patient demographic data, patient events, and patient diagnoses among other information. In the illustrated example, inputs 414 to the machine learning model are prescription related patient events and patient diagnoses, which may be received from event determination module, one or more patient information databases. The machine learning model 410 may then process the input data 414 and determine a predicted prescription adherence signal value 211D for a patient with asthma who missed their first prescription refill. In some embodiments, the machine learning model 410 may receive inputs 414 including additional factors from the patient profile and may be trained on patient information 412 containing such additional factors about patients associated with the information contained within 412. These factors would therefore be considered when determining the signal value 211D and can allow for combined effects of factors to be included in the determination of the signal value.

In some examples, a system may include one or more machine learning models trained on historical patient data which generate predictive patient signals. In some examples, the machine learning models may include statistical models and/or neural networks. In some examples, the machine learning models may be trained to predict one or more signals related to a patient based on event data related to the patient and/or patient features. The event data and patient features may be determined from patient health information, as described herein. In some examples, the machine learning models may be configured to recognize patterns in patient event data which are commonly associated with a particular patient feature or signal. In some examples, outputs of the one or more machine learning models may be used to update the urgency and/or severity score of a patient, thereby influencing the recommendations made to the user regarding what medical care or steps they may consider regarding the patient, such as suggested actions for an action plan associated with the patient. In some examples, outputs of machine learning models may be used to generate a tag for a patient profile, for example, a patient may receive a tag indicating they are at an increased probability for readmission based on the output of a ML model. Examples of signals which may be generated using ML models include, but are not limited to: a probability of readmission, a probability of comorbidities, a probability of diagnosis for a particular condition, a probability of transfer, a probability of discharge within a particular time period, among other signals. In some examples, one or more machine learning models may be used to generate suggested actions which are recommended to a healthcare provider. For example, the outputs of the one or more machine learning models may indicate that a provider should consider a specialist referral, a particular treatment, a particular prescription, a particular test, and a follow-up, among other actions for a patient. The outputs of the one or more machine learning models may be displayed to system users through a system interface, as described herein. For example, characteristics such as color, size or shape of a patient map may provide indications of outputs of the machine learning models. In some examples, outputs of the machine learning models may be displayed in one or more menus, views or other user interface elements, as described herein.

In some examples, the one or more machine learning models may be trained on historical patient data received by a system. For example, as electronic patient data is received by a system, as described herein, the electronic patient data may be stored for use in training one or more machine learning models. In some examples, electronic patient health data may be analyzed to determine whether the data should be stored for use in training of a machine learning model. For example, data may be selected for storage because it is associated with a patient having a particular patient feature, as described herein, or a patient who experienced a particular event, as described herein. For example, electronic patient health data of a particular patient may be selected for storage because the patient has been diagnosed with Alzheimer's. The stored electronic patient health data may be stored to train a machine learning model which predicts one or more signals or actions associated with patients with Alzheimer's, for example a prediction of disease progression. In some examples, data may be selected for storage because it is related to specific patient events, for example ADT events, as discussed herein, among any other patient events as described herein. In some examples, specific electronic patient health data may be selected for storage, for example, event data, test results and values, patient characteristics, and patient signals may be selected for storage. The stored electronic patient health information may be stored as one or more training datasets which may be used to train respective machine learning models. In some examples, the machine learning models may be trained using identified or de-identified datasets.

In some examples, training of the machine learning models may be performed in response to a user input to perform training of the machine learning models. In some examples, machine learning models may be trained continuously while a system is in use. In some examples, machine learning models may be trained at set time intervals, for example daily, weekly, monthly, yearly, or at any desired time interval.

In some examples, the machine learning models may be trained using one or more training techniques. In some examples, different training techniques may be used for training different machine learning models of the one or more machine learning models. Examples of techniques that may be used for training the one or more machine learning models include: gradient descent, regression analysis, and stochastic gradient descent, among other suitable training techniques. In some examples, historical patient health data may be organized into training data. The training data may include training input data and ground truth data. The training input data may include data related to patient signals, events, and characteristics, as described herein. The ground truth data may be specific patient outcomes, for example events, diagnoses, readmissions, among other patient outcomes, as described herein. In some examples, the training input data may be electronic patient health data which was input or occurred prior to the ground truth data. In some examples, the ground truth data may be selected based on the desired output of the machine learning model. For example, a machine learning model may be trained to predict the probability of readmission within 1 month following an appendectomy. The training data may be selected to include electronic patient health data for patients who had an appendectomy. The ground truth data and training input data may include data for particular patients, with the ground truth data being whether a particular patient had a readmission within 1 month of their appendectomy and the training input data may include patient features, events, and signals for the particular patient before and/or after their appendectomy.

In some examples, the outputs of the one or more ML models may be displayed to the users of systems, as described herein. In some examples, the outputs of the one or more ML models may be used in determining the urgency or severity scores for patient profiles, as described herein. In some examples, systems may filter patient profiles or displays such as patient maps and lists based on outputs of the one or more ML models, as described herein.

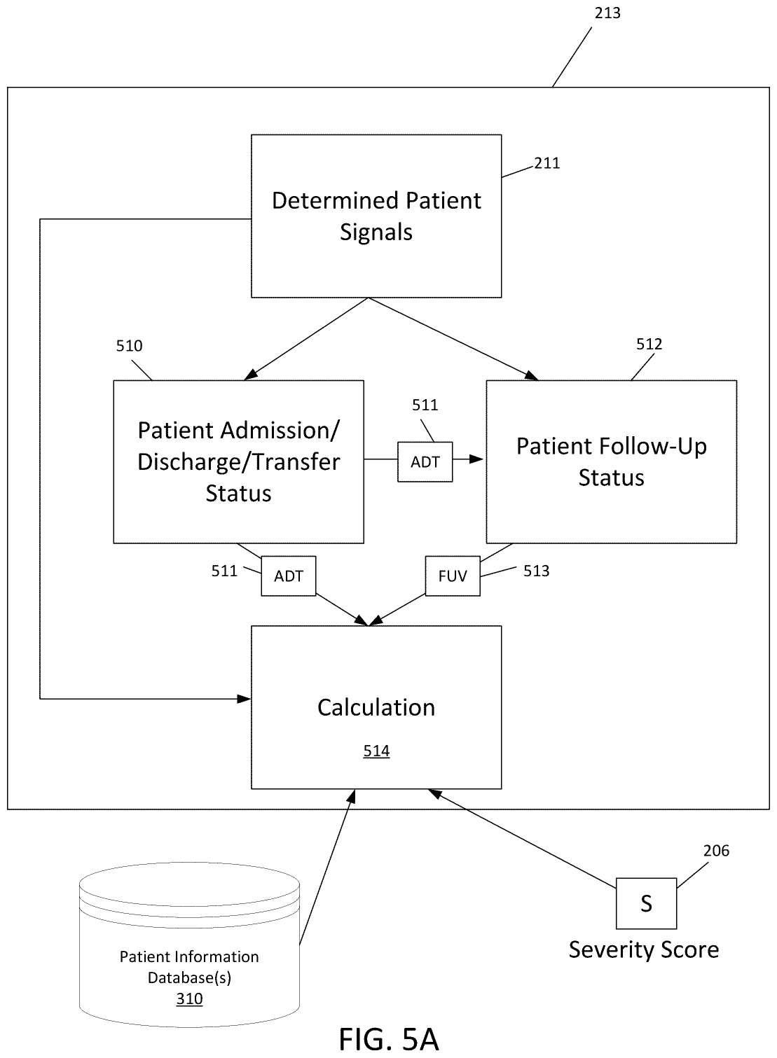

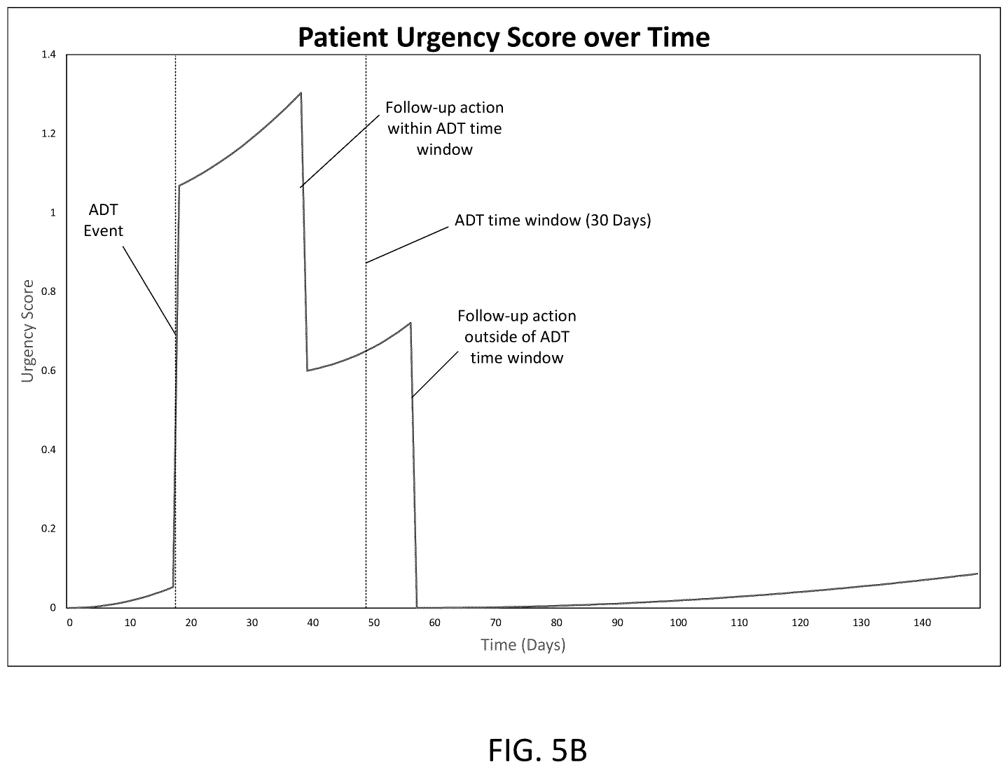

FIG. 5A illustrates an exemplary urgency scoring module 213 according to embodiments of the present invention. The urgency scoring module 213 may receive the patient signals 211 determined by the signal determination module. These signals may be passed to a patient Admission/Discharge/Transfer (ADT) Status block 510, which analyzes the determined signals 211 and outputs an ADT status 511 for the patient, indicating whether the patient has experienced one or more of an admission to a medical facility event, discharge from a medical facility event, or a transfer to a medical facility event since the previous calculation of the urgency score, or if one such event has occurred in the case the urgency score is being calculated for the first time. ADT events are important to patient urgency because they are indicative of the patient receiving medical care that is not a scheduled patient visit. These events may coincide with patient emergencies, new patient diagnoses or other medical conditions that require provider follow-up attention. Each ADT event may be considered relevant to a patient for a specified time period following the event. This time period may be set and updated by a user. The urgency score will change differently in response to actions occurring within the time period and actions occurring outside the time period. The time period may be selected based on the amount of time it may take a patient to recover from health conditions related to ADT events and allow the provider to assess the patient condition adequately after the time period has passed. The time period may also be selected based on policies that are mandated by health insurance payers, depending on the patient's insurer.

In some examples, the determined signal values may also be passed to a patient follow-up status block 512 if it is determined at the ADT block that an ADT event has occurred. The patient follow-up status block determines a status 513 reliant on if a patient experienced a follow-up or action within a specified time window of a patient ADT event. The statuses 511, 513 determined in these blocks 510, 512 are then passed to a calculation block 514, which determines a formula for the calculation of the urgency score based on the patient ADT status and the patient follow-up status and calculates the urgency score. The urgency score may be calculated based on one or more of the determined patient signals, the patient severity score 206, or information stored within patient information databases 310 related to the respective patient profile.

For example, the calculation block 514 may receive a patient ADT status 511 indicating the patient has experienced an ADT event, in combination with a follow-up status 513 indicating the patient has not experienced a follow-up within a specified time period of the ADT event. This combination of events indicates the patient has experienced an ADT event and therefore requires prompt action by a provider. Such actions may be to address the current patient condition following the ADT event or to suggest health interventions for a provider to consider with respect to the patient following the ADT event. This combination of events will result in the urgency score for the patient rising to a higher level and remaining at the higher level or increasing past the higher level until a follow-up event is recorded. After rising, the patient's urgency score may change based on one or more factors such as the patient's severity score 206, a signal indicative of a time until a recommended follow-up, and a signal indicative of a time since the patient's last visit. Each of these factors may be weighted differently in the urgency score calculation according to system presets or the preference of a user. The calculated urgency score may follow general trends based on each factor. For example, a patient's urgency score may always increase as the patient's severity score 206 increases, as a general trend.

In some examples, a patient's urgency score may change proportionally with the patient's severity score 206. In some examples, a patient's urgency score may change proportionally with the square of the patient's severity score 206. In some examples, a patient's urgency score may change proportionally with any exponent of the patient's severity score 206. A general trend for the signal indicative of a time until a recommended follow-up may be the urgency score increases as the time until a recommended follow-up decreases. In some examples, a patient's urgency score may change proportionally with the inverse of the time until a recommended-follow up. In some examples, a patient's urgency score may change proportionally with the square of the inverse of the time until a recommended-follow up. In some examples, a patient's urgency score may change proportionally with any exponent of the inverse of the time until a recommended-follow up. As a general trend, a patient's urgency score may increase as the time since the patient's last visit increases. In some examples, a patient's urgency score may change proportionally with the time since the patient's last visit. In some examples, a patient's urgency score may change proportionally with the square of the time since the patient's last visit. In some examples, a patient's urgency score may change proportionally with any exponent of the time since the patient's last visit.

For example, the calculation block 514 may receive a patient ADT status 511 indicating the patient has not experienced an ADT event, in combination with any follow-up status 513. The ADT status 511 indicating the patient has not experienced an ADT event indicates the patient's urgency score will not need to be increased for immediate attention. Another combination of patient statuses which may result in these same factors being used to determine the urgency score in the calculation block may be a patient ADT status 511 indicating a patient has experienced an ADT event and a patient follow-up status 513 indicating the patient has experienced a follow-up outside of a specified time window of the ADT event. This combination of events indicates that the follow-up occurred after a sufficient amount of time for a clinician to adequately assess patient health following the ADT event. If either of these combinations of patient statuses are received, the patient's urgency score may change based on one or more factors such as the patient's severity score 206, a signal indicative of a time until a recommended follow-up, and a signal indicative of a time since the patient's last visit. Each of these factors may be weighted differently in the urgency score calculation according to system presets or the preference of a user. The calculated urgency score may follow general trends based on each factor. The urgency score may vary with these factors as discussed above.

For example, the calculation block 514 may receive a patient ADT status 511 indicating the patient has experienced an ADT event, in combination with a follow-up status 513 indicating the patient has experienced a follow-up within a specified time period of the ADT event. This combination of events indicates sufficient time has not passed for the ADT event to not be relevant to current patient health, and therefore the follow-up which occurred within the time period is not sufficient for the clinician to adequately assess patient health, and additional actions may be required. This combination of patient statuses may result in the patient urgency score dropping from a higher value to a base value. The base value may be selected according to user preferences. In the example, the urgency score will drop because the patient has been followed-up with by a provider and therefore does not require additional attention. The patient urgency score may then increase from this base value, until a follow-up occurs outside of the specified time period of the ADT event. The patient urgency score may increase following the drop based on one or more of the following: the patient's severity score 206, a signal indicative of a time until a recommended follow-up, a time since the patient's last visit, a time since the latest of a patient admission, discharge or transfer event, the base score value, and a type of the latest patient ADT event. Each of these factors may be weighted differently in the urgency score calculation according to system presets or the preference of a user. The calculated urgency score may follow general trends based on each factor. For example, the general trends with respect to the patient's severity score 206, a signal indicative of a time until a recommended follow-up and a time since the patient's last visit may be the same general trends as discussed above. Each of the time since the latest of a patient admission, discharge or transfer event, the base score value, and the type of the latest patient ADT event may impact the formula used to calculate the patient urgency score and may directly impact the value of the patient urgency score. For example, if the latest patient ADT event was a discharge event, the urgency score may increase at a higher or lower rate in response.

A specific example of calculating the urgency score is described using the following equations, where equation 1 is a formula for calculating the urgency score based on x, the days since the last visit, and y, the days since the last ADT event. This example calculation may be utilized within urgency scoring section 212.

$$f ( x , y ) = λ {c}_{x} {x}^{2} + {c}_{y} d \qquad ( 1 )$$

The urgency score is dependent on two components, the first component being independent of patient statuses, such as those statuses 511 and 513 determined by 510 and 512 of FIG. 5A. This component is expanded according to equations 2-5 below.

$$first component = λ {c}_{x} {x}^{2} \qquad ( 2 )$$

$$λ = max ( severity score , 1 ) \qquad ( 3 )$$

$${c}_{x} = {γ}_{x}^{- 2} = min {( recommended follow up , 365 )}^{- 2} \qquad ( 4 )$$

$$first component = max ( severity score , 1 ) * min {( recommended follow up , 365 )}^{- 2} * {x}^{2} \qquad ( 5 )$$

In equations 2-5, the first factor is expanded into its components. As seen in equation 5, the first component is proportional to the severity score associated with the patient, when the severity score of the patient is greater than one. One or any other value may be selected as a minimum value for the factor associated with the severity of the patient. The first component is additionally proportional to the inverse square of the minimum of the time until recommended follow-up and 365. This will result in the first component increasing as the time until the recommended follow up decreases. 365 may be selected as a maximum value to represent situations where a recommended follow-up is an annual wellness visit, however any desired value may be used as the maximum. The first component additionally is proportional to the square of the time since the last visit, indicating the first component will increase as the time since the last visit increases.

The second component is dependent on multiple piecewise functions that determine the response of the urgency score to different patient statuses, such as those statuses 511 and 513 determined by 510 and 512 of FIG. 5A. The second component is described in more detail and expanded in equations 6-14 below:

$$second component = {c}_{y} d \qquad ( 6 )$$

The second component has two factors, d and cy. The variable d is a piecewise function expanded in equation 7 and determines the value of the second component according to different patient statuses, where a is the time window after an ADT event and h is the base score. The second component incorporates changes to the urgency score based on occurrences of ADT events.

$$d = \{ \begin{matrix}\frac{1}{{c}_{y}} if ADT event has occurred AND x > y \\ 0 if no ADT event has occured OR ( x < ( y - α ) AND y > α ) \\ {g ( x , h , {β}_{y a d j} )}^{2} otherwise\end{matrix} \qquad ( 7 )$$

The first situation occurs when patient statuses indicating a patient ADT event has occurred 511 and the time since the last visit is greater than the time since the last ADT event. This indicates the patient has not had any follow-up since an ADT event. This combination of statuses results in the patient urgency score rising, as the second component will have a value of one, giving:

$$f ( x , y ) = λ {c}_{x} {x}^{2} + 1 \qquad ( 8 )$$

The second situation occurs either when no ADT event has occurred, or when an ADT event has occurred, and the patient has received a follow-up outside of the time window a. If no ADT event has occurred, the patient's urgency score does not need to rise or drop and can continue to grow based on other factors. If an ADT event has occurred and the patient has received a follow-up outside of the time window, the patient's health was adequately assessed in response to the ADT event and there is no longer particular urgency related to the ADT event. The second situation will result in the urgency score being based only on the first component:

$$f ( x , y ) = λ {c}_{x} {x}^{2} + 0 \qquad ( 9 )$$

The third situation occurs when a patient has experienced an ADT event and a follow-up, however the follow-up occurred within the time window of the ADT event. In this situation, a patient's urgency score may lower to a base value and then increase according to a combination of factors, until the patient receives a follow-up outside of the time window. Equations 10-14 expand on factors cy and d of the second component, when the third situation occurs, where h is the base score.

$$d = {g ( x , {β}_{y adj} , h )}^{2} \qquad ( 10 )$$

$$g ( x , {β}_{y a d j} , h ) = {β}_{y a d j} \sqrt{h} + ( 1 - \sqrt{h} ) t \qquad ( 11 )$$

$${c}_{y} = {γ}_{y}^{- 2} = min {( {β}_{y a d j} , 365 )}^{- 2} \qquad ( 12 )$$

$${β}_{y a d j} = \{ \begin{matrix}max ( 0 , {β}_{y} + \frac{( ( 1 - h ) * {β}_{x} ) - {β}_{y} )}{{β}_{y}} ( y - x ) if x < y \\ {β}_{y} otherwise\end{matrix} \qquad ( 13 )$$

$${β}_{y} = \{ \begin{matrix}10 if ADT event is a discharge \\ ( 1 - h ) * {β}_{x} otherwise\end{matrix} \qquad ( 14 )$$

The above example equations may be used to calculate a patient's urgency score. An example of a patient's urgency score changing over time, in response to different events and actions, is shown in FIG. 5B. At the start of the graph of FIG. 5B, the urgency score is low and growing. The urgency score may be growing due to an upcoming recommended follow-up, a severity score of the patient or a time since the last patient visit changing. This would correlate with patient statuses indicating the patient had not experienced an ADT event, or the patient had previously experienced an ADT event and a follow-up for the event outside of the time window of the event.

In this example, the patient urgency score rises just before day 20. This may be in response to the patient experiencing an ADT event at this time and no patient visit has occurred since the ADT event. In this example, the patient urgency score increases by one, and this may indicate to a user that the respective patient requires immediate action.

The patient urgency score continues to increase following the rise until a drop just before day 40 to a base value of 0.6. This may occur in response to the patient experiencing a follow-up within the time period of the ADT event. In this example the time period of the ADT event is 30 days. Following the drop before day 40, the urgency score increases. The rate of increase may be based on a severity score of the patient, a time until a recommended follow-up, a time since a last visit or any other factor as previously discussed. The urgency score grows until it drops just before day 60. This drop may be in response to the patient experiencing a follow-up outside of the time period associated with the ADT event. In this example, the time period would end just before day 50. After dropping, the patient urgency score may depend on a scheduled patient visit, a severity score of the patient or a time since the last patient visit, as previously discussed.

In some examples, signals and events determined for patients may additionally be used in preparing a suggested action plan associated with the patient. An action plan includes specific actions that a provider may consider performing in response to signals and events associated with the patient. Each action within an action plan may have one or more features associated with it, such as a type of action, a time of action, a reason for the action, an action category, notes on the action and an action status. Examples of action statuses include: New—the action is being seen for the first time or no activity has been taken to resolve the action, Hold—the action has been acknowledged by the user however no activity has been taken to directly resolve the action, Completed—the user has completed the action, Dismiss—the user has decided no activity is necessary to resolve the action. A user may change the status of an action via inputs to the system, for example, inputs to a GUI of the system. Changes to action statuses are provided may be used by the system as patient health information inputs and may be identified as events and result in signal changes within the system.

Determining suggested action plans, severity and/or urgency scores for patients as described herein, provides advantages over traditional patient management systems because it gives healthcare providers an indication of patient need which is based on multiple factors besides their most recent medical event, their reason for admission to a medical facility, patient self-assessment or patient decisions on informing providers on conditions. The additional factors may include, but are not limited to patient features, patient events, patient signals and patient severity scores, as described herein. This additional patient data provides healthcare providers with a more informed understanding of patient conditions and can provide data to facilitate more informed medical decisions by providers.

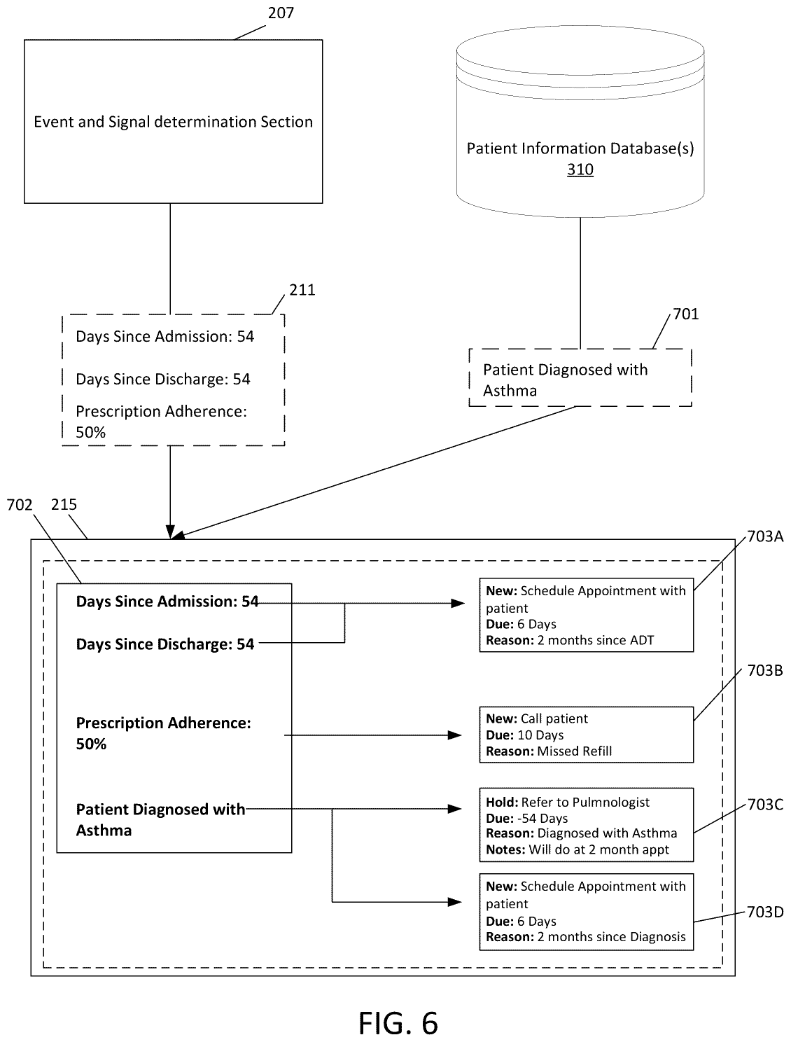

FIG. 6 illustrates an example of an action plan section of a system. The action plan section may receive information related to patient signals 211, and event data 209 from an event and signal determination section of the system such as section 207 of FIG. 2. The action plan section may also receive patient health information related to provider actions or any other information maintained by the system within patient profiles. In this example, the action plan section may receive signals 211, such as those generated in FIG. 4A. The action plan section may also receive information that the patient has been diagnosed with asthma 701 from patient information databases 310. Each of these inputs 702 may result in one or more actions being generated by the action plan module. The generated actions may be considered by system users, for example, medical providers, for patient care. A single input to the action plan module may result in a single action being generated, multiple actions being generated, or no actions being generated. In the instance no actions are generated by a single input, multiple inputs may be required to generate one or more actions. Examples of these cases are shown in FIG. 6.

The signals of days since admission and days since discharge together result in a single action 703A being generated. This may be because these are both signals related to ADT events which have the same value. The generated action 703A has a new status, indicating the action may not have been viewed by the provider. The action has a time of six days until it is due, this time may be generated automatically based on rules associated with action types, may be determined by provider preference, or may be input by the provider. The reason for the action is because two months have passed since the last ADT event.

The prescription adherence signal results in a single call patient type action 703B being generated for provider consideration. This action 703B is also a new action, has an associated time of ten days, and is generated because the patient missed a refill.

The health information indicating the patient has been diagnosed with asthma results in two actions being generated 703C and 703D, for provider consideration. The first action 703C, refer to pulmonologist, is an action on hold, indicating a provider has viewed the action and decided it will be addressed at a future date. The provider may have performed this immediately following the patient's diagnosis, knowing the patient will have a follow-up soon after. The action status may be provided to the action plan section from patient health information provided from patient information databases or other sources not depicted in FIG. 6.

In some examples, the provider may add one or more notes associated with the action when selecting to put the action on hold. In this example the provider indicated they will refer the patient to a pulmonologist at the 2-month appointment. The time for the action may be displayed as negative because it is overdue. Alternatively, the time may be displayed as 54 days overdue, or in any way suitable to convey the due date has passed. The health information indicating the patient has been diagnosed with asthma additionally results in a schedule appointment with patient action being generated. In this case the action is a new action with a time of six days until it is due because it has been two months since the diagnosis.

Action 703D is a new scheduled appointment with patient status which has a time of six days and has been generated because it has been two months since the patient diagnosis. This action is also generated by the data indicating the patient was diagnosed with asthma. The system may always generate two or more events in response to a particular event or input data.

In some examples, the actions generated at the action plan module may be added to a patient's existing action plan or may become the patient's action plan in the case the patient does not have an existing action plan. Action plans may be sent to respective patient profiles for providers to access, consider, modify and/or complete.

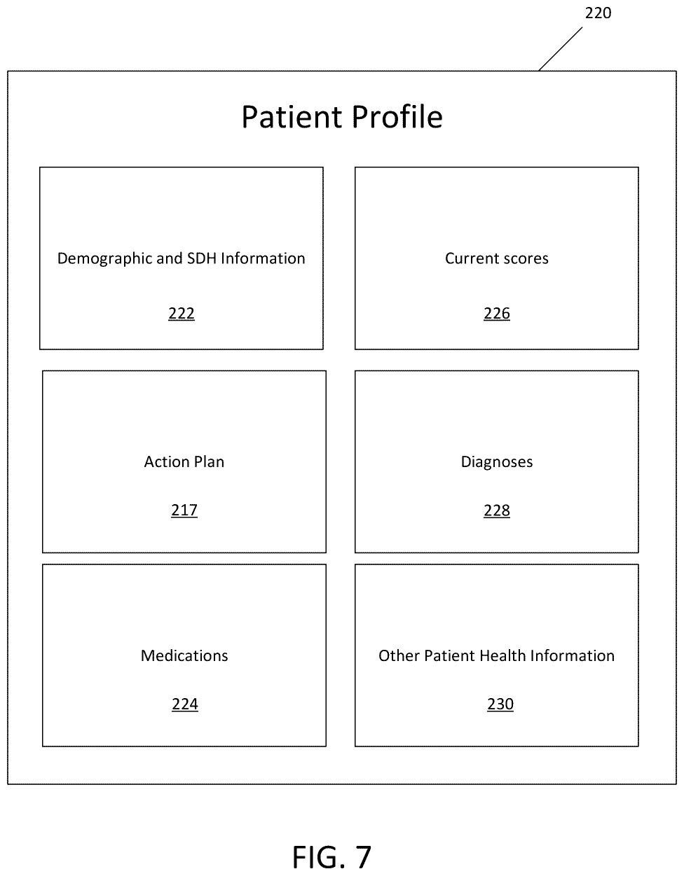

FIG. 7 illustrates a representation of a patient profile 220 and information which may be stored by a system within a patient profile. The patient profile may receive patient information from one or more sources as discussed herein, for example the patient profile may receive information from obtained patient health information, a severity scoring section, an urgency scoring section, and an action plan section of the system. The patient profiles may be stored by a system in a datastore, for example a database or other form of non-transitory storage. In some examples, the patient profiles may be stored external to a system, for example in a cloud database which is accessible by the system. The patient profiles may be stored using one or more data structures, as described herein.

In some examples, the patient profile may contain general patient information 222 including patient demographic information, and patient SDOH information. Patient demographic information within 222 may include: an age of the patient, the gender of the patient, an ethnicity of the patient, a date of birth of the patient, and the patient's name. SDOH information within 222 may include one or more of: a patient income, a patient location, a patient education level, a patient employment status, a patient family condition, one or more patient behaviors, and a measure of patient health care access. The patient profile may contain patient scores 226, including a severity score calculated by the severity scoring module and an urgency score calculated by the urgency scoring module. The patient profile may contain a patient action plan 217, which contains one or more actions and associated action information generated by action plan module, for provider consideration. The patient profile may contain current and past patient diagnoses 228. These diagnoses 228 may be received in obtained patient health information, or may be input directly by a user of the system, in response to a new patient diagnosis. The patient profile may contain current and past patient medications 224. These medications 224 may be received in obtained patient health information or may be input directly by a user of the system, in response to a new patient prescription. Other patient information within 230 may include a height of the patient, a weight of the patient, a Medicare ID or similar unique identifier of the patient, a patient insurer, a patient cohort or category, Medicare Risk Adjustment Factor of the patient, last patient touchpoint, last patient visit, last update of claims data related to the patient, and the primary care provider of the patient, among other health information received by or input into the system.

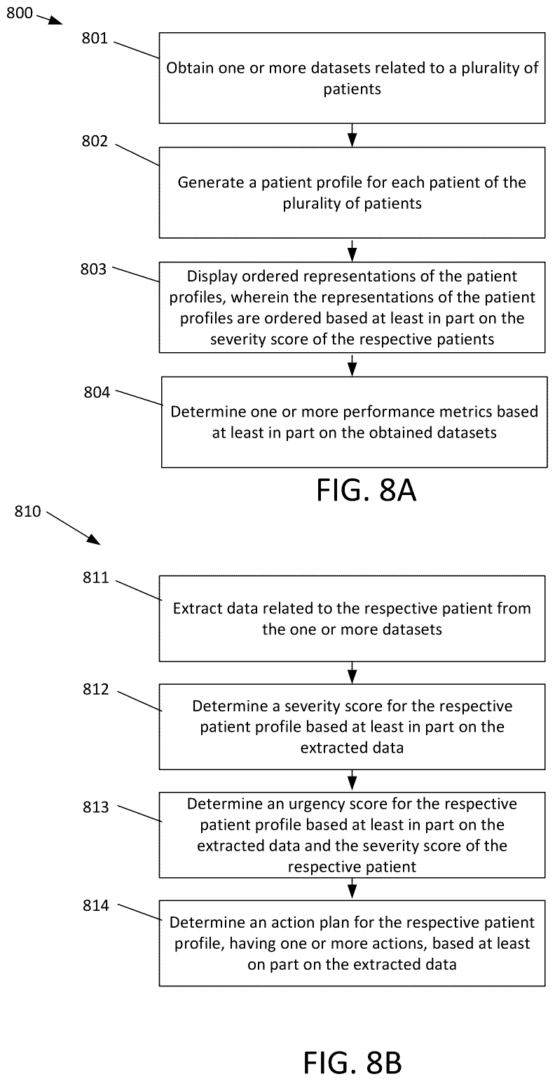

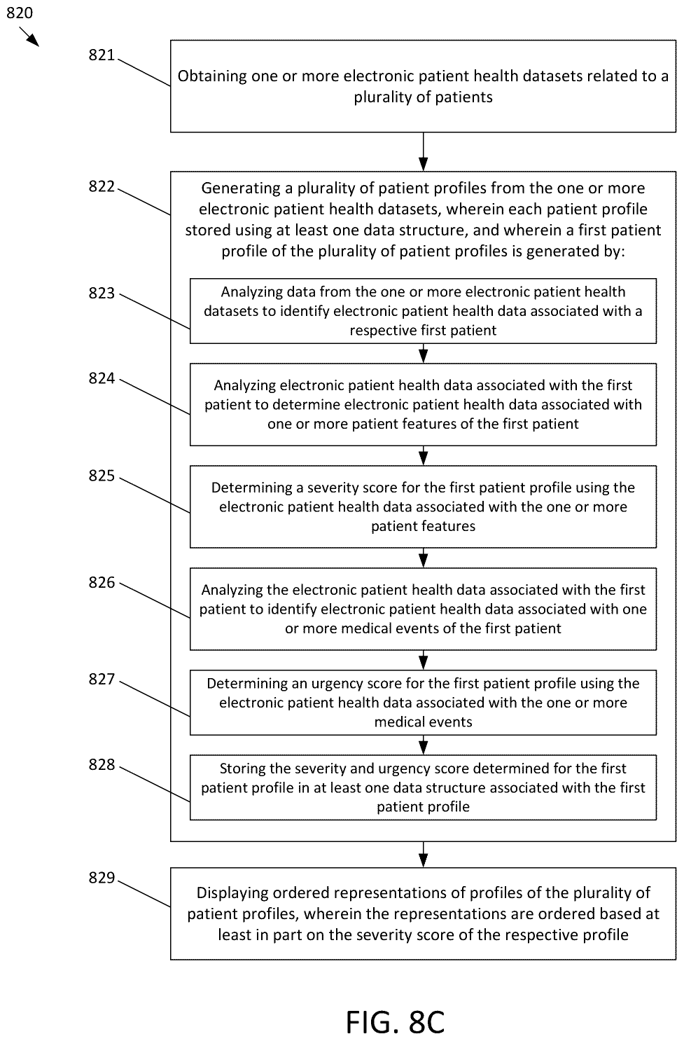

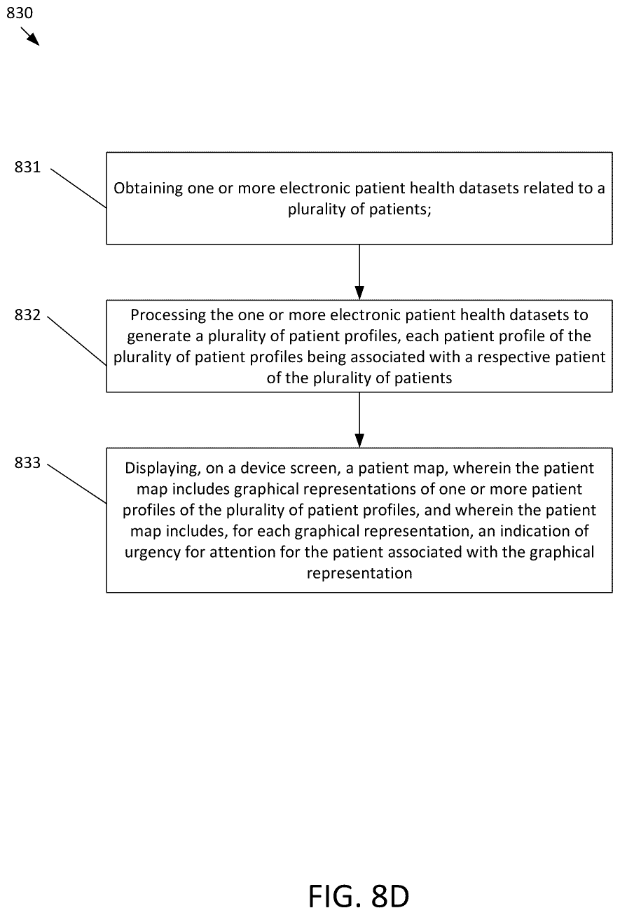

FIG. 8A illustrates a process 800 for preparing patient profiles and displaying representations of patient profiles. Process 800 may be performed by a system such as system 110 of FIG. 1. Process 800 begins at step 801, in which one or more datasets related to a plurality of patients are obtained. The one or more datasets may be obtained from one or more of: Electronic Health Records (EHR) vendors, Electronic Medical Records (EMR) vendors, Insurance Providers, System Data and Health Information Exchanges (HIE), among other data vendors. The one or more obtained datasets may contain data in any suitable format as discussed herein. The one or more obtained datasets may be obtained by the system using a push-based process, a pull-based process or a combination of push and pull-based processes as discussed herein.

In some examples, after obtaining the one or more datasets, process 800 proceeds to step 802, in which a patient profile is generated for each patient of the plurality of patients. Specific steps for generating the patient profiles are discussed below with relation to FIG. 8B. Each patient profile of the plurality of patient profiles may include information as discussed with relation to FIG. 7. This information may be associated with a particular patient profile by the system.

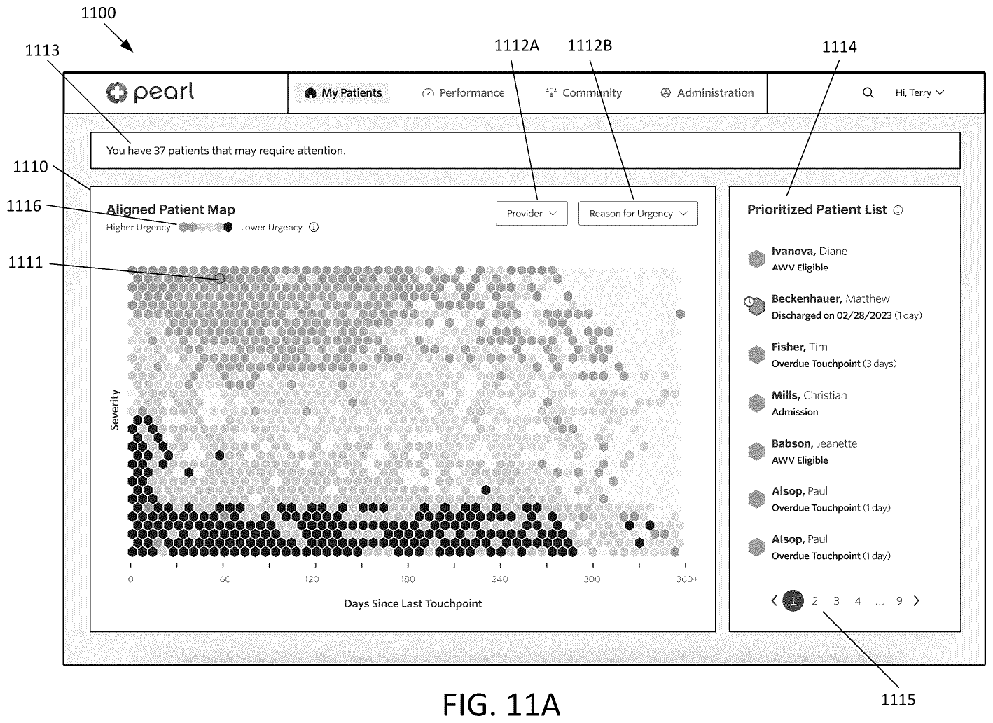

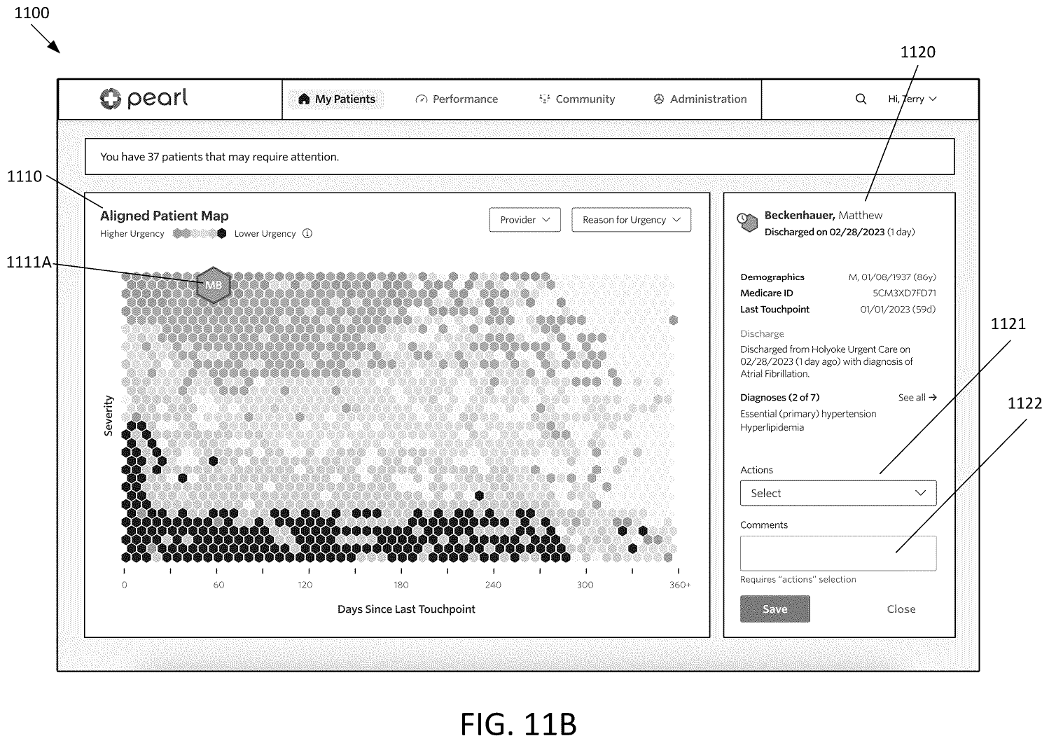

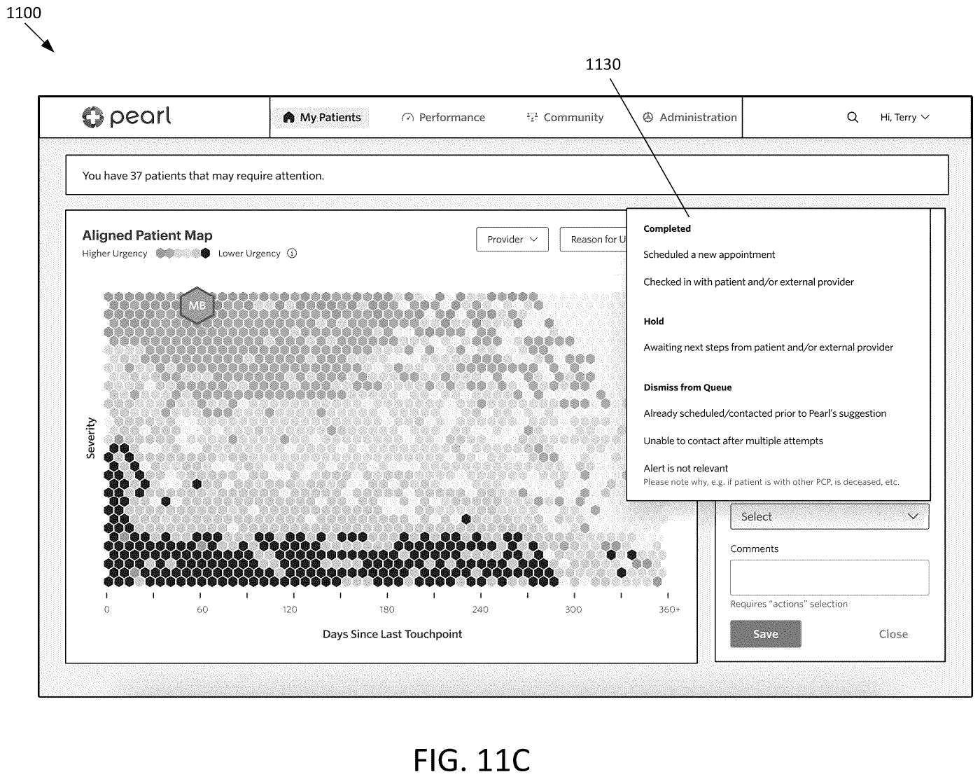

After performing step 802, process 800 proceeds to step 803, in which ordered representations of the patient profiles are displayed, based at least in part on the severity score of the respective patients. The system may display representations of the patient profiles using a Graphical User Interface (GUI) shown on a display. The display may be internal or external to the system. The GUI is discussed in more detail in relation to FIGS. 11A-13. As described herein, displaying ordered representations of patient profiles based on at least the severity scores of associated patients provides benefits over conventional systems because the patients are ordered based on a predicted benefit of proactive intervention, which allows system users to understand the scope of needs of their patients and more effectively and efficiently prioritize patients for care.

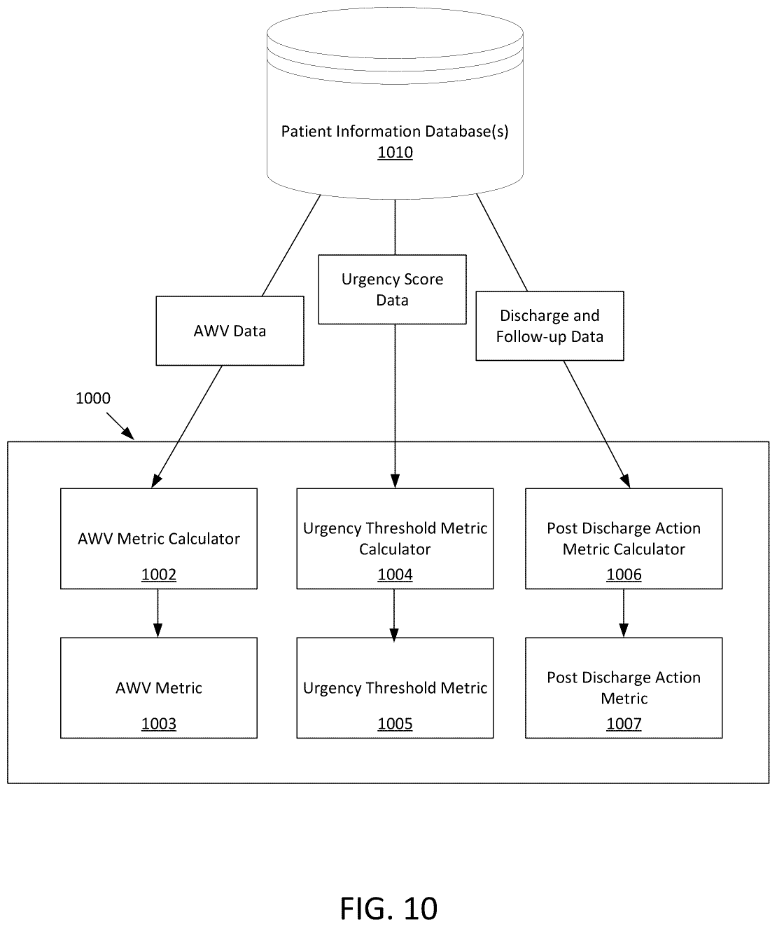

Process 800 may optionally perform step 804, in which one or more performance metrics are determined based at least in part on the one or more datasets or the generated patient profiles. These performance metrics may allow users to analyze their performance with regard to patient care and may provide an indication as to the level and quality of care provided to the patient group treated by the users of the system. The determination of performance metrics is discussed in more detail with respect to FIG. 10.

FIG. 8B illustrates a process 810 which may be performed by a system to generate a patient profile related to a respective patient. Process 810 begins at step 811, in which data related to the respective patient is extracted from the one or more datasets. Step 811 may involve analyzing the one or more datasets to determine data related to a specific patient of the plurality of patients. Step 811 may additionally store one or more pieces of information related to the patient within the respective patient profile. The one or more pieces of information may include patient demographic information, patient SDOH information, patient diagnoses, patient medications and other patient information, as discussed with relation to FIG. 7.

After step 811, process 810 proceeds to step 812, in which a severity score for the respective patient profile is determined based at least in part on the data extracted in step 811. The patient severity score may be calculated according to the techniques as described in relation to FIG. 3.

After step 812, process 810 proceeds to step 813, in which an urgency score for the respective patient profile is determined based at least in part on the data extracted in step 811 and the severity score calculated in step 812. The patient urgency score may be calculated according to the techniques as described in relation to FIGS. 4A-6A. For example, step 813 may involve determining a plurality of events from the data extracted in step 811. Step 813 may additionally involve determining from the plurality of events, a plurality of signals related to the respective patient. The plurality of events and signals may be processed with the patient severity score in step 813 to determine the urgency score associated with the patient profile.

Process 810 may also include step 814, in which an action plan is determined for the respective patient profile. The action plan may include one or more actions which are determined based at least in part on the extracted data. The one or more actions of the action plan may also be determined based on patient signals or determined events. The action plan may be determined using the techniques described with relation to FIG. 6, and may be provided to users of the systems, for example healthcare providers, for consideration.

In some examples, process 810 may additionally be performed to re-calculate severity and urgency scores associated with a patient profile in response to the system receiving new patient health information.

FIG. 8C illustrates a process 820 which may be performed by a system to generate and display patient profiles, in accordance with some embodiments of the technology described herein. The process 820 begins with step 821, in which one or more electronic patient health datasets related to a plurality of patients are obtained. The one or more datasets may be obtained as discussed herein, for example as discussed with regard to FIG. 1 or FIG. 2, for example the electronic patient health datasets may be obtained from external systems 120 as patient health information, as discussed with regard to FIG. 1.

Process 820 may then proceed to step 822, in which a plurality of patient profiles is generated from the one or more electronic patient health datasets. Each patient profile is stored using at least one data structure. Examples of data structures for storing patient profiles are described herein. In some examples, step 822 may be performed by a system as described herein, for example system 200 of FIG. 2.

Steps 823-828 are performed for generating a first patient profile associated with a first patient of the plurality of patients. In some examples, these steps may be performed to generate patient profiles for other patients in the plurality of patients. The electronic patient health data may be analyzed as described herein. In step 823, data from the one or more electronic patient health datasets are analyzed to identify electronic patient health data associated with a respective first patient. Step 823 may be performed within a system as described herein, for example using one or more modules of system 200 of FIG. 2, such as severity scoring section 202. In some examples step 823 may be performed using a data extraction module, such as 203 as discussed with regard to FIGS. 2 and 3.

In some examples, the process may then proceed to step 824, in which electronic patient health data associated with the first patient is analyzed to determine electronic patient health data associated with one or more features of the patient. The electronic patient health data may be analyzed as described herein. Step 824 may be performed within a system as described herein, for example using one or more modules of system 200 of FIG. 2, such as severity scoring section 202. In some examples step 824 may be performed using a data extraction module, such as 203 as discussed with regard to FIGS. 2 and 3.

In some examples, the process may then proceed to step 825, in which a severity score is determined for the first patient profile using the electronic patient health data associated with the one or more patient features. The severity score may be determined as described herein. For example, the severity score may be determined by a severity scoring module such as 205, as described herein.

In some examples, the process may then proceed to step 826, in which electronic patient health data associated with the first patient is analyzed to identify data related to one or more medical events of the first patient. Medical events may include any medical events as described herein. Step 826 may be performed within a system as described herein, for example using one or more modules, of system 200 of FIG. 2, such as event and signal determination section 207. In some examples step 826 may be performed using an event extraction module, such as 208 as discussed with regard to FIGS. 2 and 3.

In some examples, the process may then proceed to step 827, in which an urgency score is determined for the first patient profile. The urgency score may be determined as described herein, for example as described with regard to FIGS. 2, 4A, 4B, 5A and 5B. Step 827 may be performed within a system as described herein, for example using one or more modules of system 200 of FIG. 2, such as urgency scoring section 212. In some examples step 827 may be performed using an urgency scoring module, as described herein, such as 213 as discussed with regard to FIGS. 2 and 5A.

In some examples, the process may then proceed to step 828, in which the severity and urgency scores for the first patient profile are stored using one or more data structures. The severity and urgency scores may be stored as described herein, using any suitable data structures.

In some examples, steps 823-828 may be performed for each patient of the plurality of patients to generate the plurality of patient profiles.

In some examples, the process may then proceed to step 829, in which ordered representations of the plurality of patient profiles are displayed. The ordered representations may be ordered based at least in part on the severity score of the respective profiles. In some examples, the profiles may be displayed as a part of a GUI, as described herein.

FIG. 8D illustrates a process 830 which may be performed by a system for displaying patient profiles, in accordance with some embodiments of the technology described herein. Process 830 begins at step 831, in which one or more electronic patient health datasets related to a plurality of patients are obtained. The one or more datasets may be obtained as discussed herein, for example as discussed with regard to FIG. 1 or FIG. 2, for example the electronic patient health datasets may be obtained from external systems 120 as patient health information, as discussed with regard to FIG. 1.

In some examples, the process may proceed to step 832, in which the one or more patient health datasets are processed to generate a plurality of patient profiles. Each patient profile is associated with a respective patient of the plurality of patients. The patient profiles may be generated as described herein.

In some examples, the process may proceed to step 833, in which a patient map is displayed on a device screen. The patient map includes graphical representations of one or more patient profiles of the plurality of patient profiles, and includes, for each graphical representation, an indication of urgency for attention for the patient associated with the graphical representation. The patient map may be displayed on a device within a medical setting, or any suitable device, as described herein. The patient map may be displayed as a part of a GUI, as described herein.

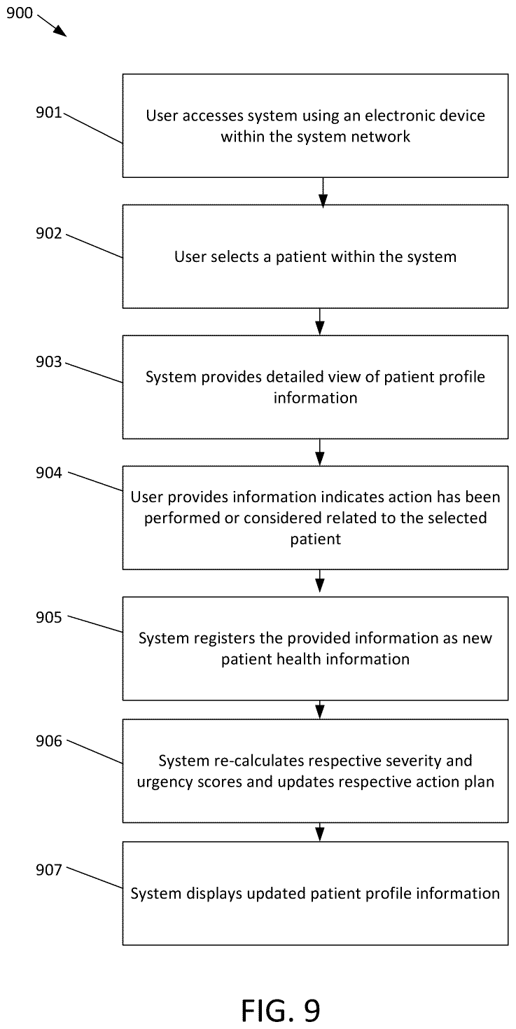

FIG. 9 illustrates an example process 900 which may be followed during the implementation and use of a system. The steps of process 900 may be performed by a system, such as system 110 of FIG. 1 or may be performed by a user of the system. Process 900 begins at step 901, in which a user accesses the system using an electronic device within the system network. At step 902, the user may select a patient within the system. The patient may be selected based on one or more factors associated with the patient such as an urgency score of the patient. In response to the patient selection, the system may provide a detailed view of information related to the patient from the associated patient profile to the user in step 903. In step 904, the user may indicate to the system an action has been performed or considered with relation to the selected patient. In step 904, the user may access, consider, modify and/or complete an action by specifying a type of action, an action status, a time of action or any other feature of an action of a system such as those discussed with relation to FIG. 6.

In response to the indication an action has been performed or considered, in step 905, the system may register the indicated action as new patient information and will proceed to step 906. In step 906, the system may re-calculate the severity score and urgency score associated with the respective patient profile and may update the action plan of the respective patient profile. Step 906 may involve the techniques described within FIGS. 2-6 for the re-calculation of scores and updating of the action plan. Process 900 may then proceed to step 907, in which the system displays updated patient profile information to the user. The updated patient profile information displayed in step 907 may include an indication of the patient severity score, an indication of the patient urgency score and an indication of a patient action plan.

FIG. 10 depicts a data processing section 1000 of the system which may be used to calculate one or more healthcare performance metrics. In some examples, the healthcare performance metrics may be used by the healthcare location, locations or network to which the system is deployed to analyze the level of care which is being provided. In some examples, healthcare performance metrics such as a value-based care metric may be used in the determination of compensation for medical care.

In some examples, a first performance metric which may be calculated is a percentage of patients who have completed or scheduled an annual wellness visit (AWV) 1003. The metric may be calculated by AWV metric calculator 1002. An AWV is a patient visit that should occur once per year in which a provider may analyze the overall health of the patient. AWVs are important indicators for patient health and outcomes.

In some examples, AWV calculator 1002 may determine the number of patients having scheduled an AWV by analyzing information contained within patient profiles, which may include signals indicative of a time until AWV. This information may be obtained from patient information databases 1010, which may be internal or external to the system. After determining the number of patients having scheduled an AWV, this may be compared to the total number of patients to determine a percentage of patients having scheduled an AWV. The number of patients having completed an AWV may be determined by analyzing information contained within patient profiles, which may include a signal of time since last AWV or a patient event or action indicating an AWV has occurred in the past year. After determining the number of patients having completed an AWV, this may be compared to the total number of patients to determine a percentage of patients having completed an AWV. The percentage of patients having completed an AWV may be added to the percentage of patients who have scheduled an AWV in order to determine a total percentage of patients who have scheduled or completed an AWV 1003. This total percentage may be compared to a goal percentage. The goal percentage may be set by the system, or by a user to represent the goals of the healthcare location, locations or network to which the system is deployed.

In some examples, a second performance metric which may be measured by the system is the average percentage of patients having an urgency score over a threshold score 1005. This provides an indication of how quickly patient urgency is responded to within the healthcare location, locations or network to which the system is deployed. This metric may be determined by urgency threshold metric calculation 1004 based on urgency score data received from one or more patient information databases 1010. This metric may measure the percentage over a specified time period, for example, a one-month period. Other time periods may be used, for example a one-week, multi-week, multi-month or yearlong time period may be selected. A healthcare location, locations or network may desire to keep the percentage of patients having an urgency score greater than a threshold score below a goal percentage, which indicates patients receive attention to address their medical needs. This threshold score metric 1005 may be determined by analyzing stored patient data at multiple time points within a certain time period. At any given time, percentage of patients having an urgency score greater than a threshold score may be determined by analyzing patient urgency scores to identify a number of patients having an urgency score over the threshold score and comparing this number to the total number of patients to determine a percentage of patients having an urgency score greater than the threshold. For example, if the metric is to be calculated across a specified time period, a percentage may be calculated for each day within the time period and averaged to give the final average percentage of patients having an urgency score greater than a threshold score metric 1005. Alternatively, a percentage may be calculated based on a subset of days within the time period, for example, every other day, every third day, every fourth day, one day per week or any other time period.

In some examples, a third performance metric which may be calculated is a post-discharge action metric 1007. This metric may be calculated by post discharge action metric calculator 1006, using discharge and follow-up data from one or more patient information databases 1010. The post discharge action metric 1007 may indicate what percentage of discharge events received a follow-up action within a given time period, over a specified period. For example, the time period for follow-up may be within two days of the discharge event, however any suitable value may be chosen on the scale of single days or tens of days. The metric 1007 may measure the percentage over a specified time period, for example a one-month period. Other time periods may be used, for example a one-week, multi-week, multi-month or one year time period may be selected. This metric 1007 may be calculated by analyzing stored patient health information for every patient experiencing a discharge event within the given time period. After this set of patients are identified, the number of patients who received a follow-up within the given follow-up period are identified and this number is compared to the total number of patients experiencing a discharge event to give a percentage representing the post-discharge action metric 1007. A healthcare location, locations or network may desire to keep the post-discharge action metric 1007 greater than a threshold score, which indicates patients are receiving the necessary follow-up care after discharge events.

In some examples, the data processing section 1000 may calculate additional performance metrics. For example, the data processing section may calculate a value-based care metric, which is indicative of a value of healthcare generated due to the deployment of the system, for example system 110. In some examples, the value based care metric may be a financial value, for example in dollars, of the value of healthcare generated due to the deployment of the system.

In some examples, the system may display these metrics to users, so users can adequately assess their performance in meeting patient care standards and their patient care goals. Improvements in these metrics may result in improved patient outcomes for healthcare locations.

FIG. 11A illustrates an example of a graphical user interface 1100 for displaying data related to multiple patient profiles within a healthcare network. The graphical user interface (GUI) 1100 may be displayed to healthcare providers in order to provide an overview of the current conditions of all patients and which patients require prompt follow-up. The GUI may be shown on the display of a digital device such as those discussed in relation to FIG. 1. This may be used to prioritize patient care and ensure all patients requiring action receive proper care.

The GUI 1100 may include a patient map 1110 which displays information related to all patients, or a subset of patients within a healthcare network. The patient map may represent each patient using a symbol 1111 to indicate one or more factors associated with the patient. In the example of FIG. 11A, each patient is represented by a hexagon on the patient map 1110. Different features of the symbols may represent the one or more factors associated with the patient. For example, a vertical position, horizontal position, size, shape, and/or color of the symbols may be used to represent one or more factors associated with the patient. In the example of FIG. 11A, the color of each hexagon corresponds to the urgency score of the corresponding patient. A scale 1116 is provided on GUI 1100 to illustrate the relationship between the color of the hexagon and the urgency score of the respective patient.

In some examples, the vertical location of each hexagon on GUI additionally corresponds to the severity score of the respective patient. Patients with higher severity scores have hexagons positioned higher on the patient map than patients with lower severity scores. The horizontal position of the hexagons on the patient map correspond to the time since the last touchpoint with the respective patient, with hexagons related to patients having more recent touchpoints positioned on the left of the patient map. The GUI may provide one or more filters 1112 for users to change the display of patients on the patient map. For example, the GUI may provide a filter 1112A which displays the hexagons on the patient map related to a specific provider. This filter may allow providers to view only their patients to determine their workflow. This feature may be useful in an embodiment where the system is located centrally in a healthcare location where it may be accessed by multiple providers. The GUI 1100 may additionally provide filters 1112B for the reason for urgency, which may allow providers to view patients which have common reasons for urgency. One example is patients with increased urgency due to recent ADT events may be of particular concern to a provider, and therefore the provider may filter patients by this metric.

In some examples, a user may navigate the GUI 1100 in a variety of ways, depending on the device the GUI is displayed on. For example, the GUI 1100 may be displayed on a touch screen device, such as a tablet, or a touch screen computer. A user may navigate the GUI 1100 on a touch screen device by tapping on different portions of the GUI, and the GUI 1100, in response to the tapping will change the information currently being displayed. In other examples, the GUI 1100 may be displayed on a device which has a cursor that can be navigated through an external mouse, a touch pad, a button or any other suitable device. A user may navigate the cursor to a portion of the GUI 1100 and click on that portion to make a selection or input to the GUI 1100. In response, the GUI 1100 will change the information currently being displayed.

In some examples, the GUI 1100 may additionally provide one or more alerts 1113 to providers about the status of patients within the healthcare network. In the example of FIG. 11A, an alert is provided stating 37 patients are predicted to require proactive attention. Alerts may be provided based on the urgency score associated with a patient or the action plan associated with a patient.

In some examples, the GUI 1100 may additionally provide a prioritized patient list 1114, which allows providers to view information related to individual patients, organized based on the urgency and need of patients. Within the prioritized list 1114, the patients are organized based on their respective urgency scores, with patients having higher urgency scores displayed first in the prioritized patient list 1114. The prioritized patient list 1114 may display information related to patients including a patient name, a reason for urgency and a time associated with the patient, as shown in the prioritized patient list. Additional patient information may be displayed in the prioritized patient list 1114, for example, a last patient touchpoint, a type of event associated with the patient, and a diagnosis associated with the patient among other information maintained by the system. The prioritized patient list 1114 may be navigated by selecting a page of the prioritized patient list using page selector 1115.

In some examples, the GUI 1100 may provide additional views of patient information. FIG. 11B illustrates an example of a GUI 1100 displaying a patient summary 1120 alongside a patient map. A provider or user may prompt the GUI to display a patient summary 1120 by selecting a hexagon 1111 associated with the patient from the patient map 1110 or by selecting a patient from a prioritized patient list 1114. The patient summary 1120 may be displayed on a side of the GUI 1100, next to the patient map 1110. When a patient is selected, the patient summary 1120 may replace the prioritized patient list in the GUI 1100. The patient summary 1120 may display information related to the patient from the associated patient profile, for example, a patient name, a reason for patient urgency, patient demographic information, a patient identifier, a last patient touchpoint, information about the most recent patient event, and patient diagnoses. Additional patient information maintained by the system may be displayed in the patient summary 1120. When a patient is selected for the display of the associated patient summary 1120, the symbol associated with the patient in the patient map may change in appearance to illustrate which patient summary is displayed. In the example of FIG. 11B, the hexagon 1111A associated with the selected patient is enlarged, has a dark border around it and has patient initials displayed at its center. The patient summary may additionally include an actions section 1121 where a provider may perform one or more actions associated with the patient. A provider may additionally add a comment to the patient profile related to an action performed by the provider in the comment portion 1122 of the actions section 1121.

FIG. 11C illustrates the actions 1130 which a provider may select, from the patient summary. These actions include actions to indicate the provider has addressed the patient need, shown as completed, actions to indicate the provider is aware of patient need but cannot address the need at this time, shown as hold, and actions to indicate the patient urgency is no longer relevant to the provider, shown as dismissed. Completed actions may include scheduled a new appointment and checked in with patient and/or external provider. Hold actions may include awaiting next steps from the patient and/or external provider. Dismiss from queue actions may include already scheduled/contacted prior to urgency indication, unable to contact after multiple attempts, and alert is not relevant.

In some examples, patient actions input into the system may be used to update information within patient profiles. These patient actions may be inputted to the system as patient health information and used to update information within the patient profile. For example, scheduling an appointment may cause an urgency score for a patient to decrease as a follow-up has occurred.

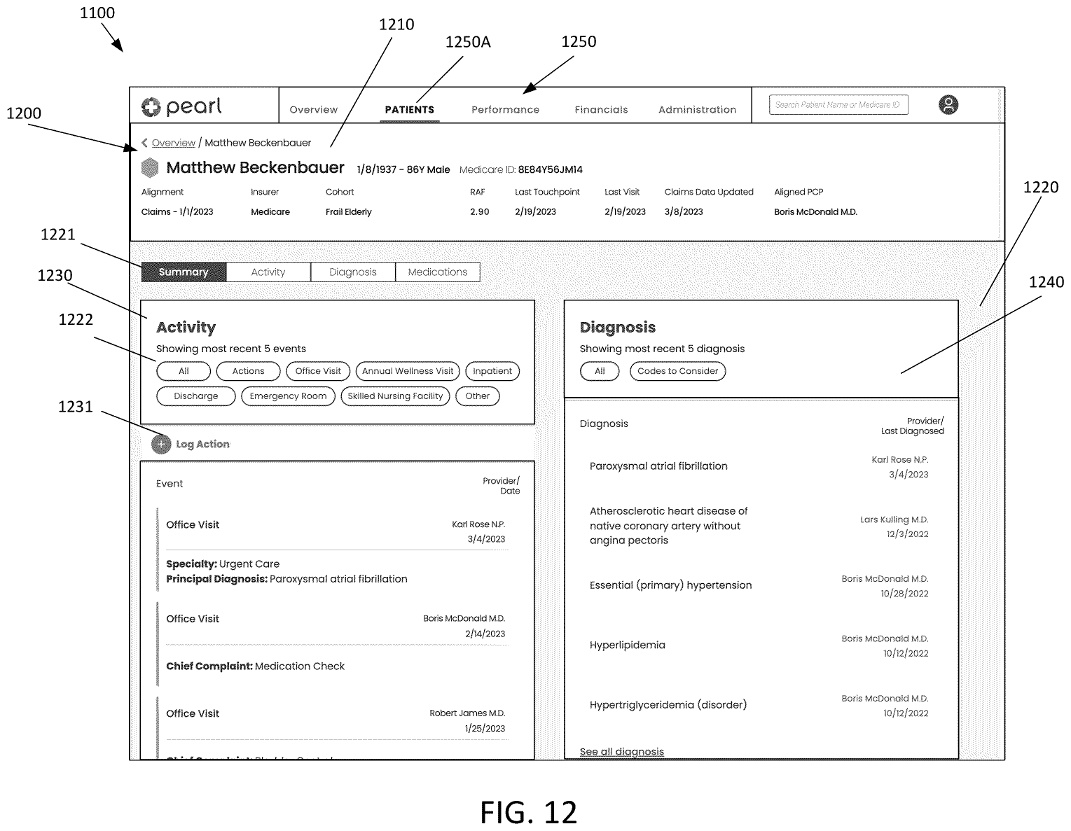

In some examples, providers may also desire to view a more complete view of a patient. FIG. 12 illustrates a detailed patient view which may be provided by a GUI 1100. The detailed patient view displays more information than the patient summary view of FIG. 11B.

In some examples, the detailed patient view 1200 may include a section which displays general patient information from a patient profile 1210, shown at the top portion of GUI. The general patient information may include the patient's name, date of birth, age, Medicare ID or similar unique identifier, insurer, cohort or category, Medicare Risk Adjustment Factor or another indication of the severity score, last touchpoint, last visit, last update of claims data related to the patient, and the primary care provider of the patient. This data may be useful for a user to understand a general view and outlook of the patient before analyzing the additional patient information displayed below on the GUI.

In some examples, the lower section 1220 of the detailed patient view may include information related to patient activity, diagnosis and medications. As displayed in FIG. 12, a summary view is shown, where a view of the patient Activity and Diagnosis are visible. The patient medications may additionally be shown in this view and could be displayed alongside the Activity and Diagnosis views or could be displayed below the Activity and Diagnosis views and accessed by scrolling to the view. A view selector 1221 may be provided by GUI 1100 in order for the user to select between Summary, Activity, Diagnosis and Mediation views by selecting the button corresponding to the desired view. These views may be accessed through additional GUI selections, for example selecting the See All Diagnoses button may display the diagnosis view. The currently selected view may be identified by having a different color than the other buttons, Summary in FIG. 12. The Summary view displays the most recent information available related to patient Activity, Diagnosis and Medications, in this example the most recent five events, diagnoses or medications, however any number may be displayed. Selecting any one of the Activity, Diagnosis and Medication views will result in only data related to that view being shown in the lower section of the detailed patient view.

In some examples, each of the Activity, Diagnosis and Medication views may include one or more filters 1222 which a user may select from to focus the data displayed with relation to that view. For example, the Activity view includes nine filters 1222 which a user may select from: All, Actions, Office Visit, Annual Wellness Visit, Inpatient, Discharge, Emergency Room, Skilled Nursing Facility, and Other. Selecting any one of these filters may change the data displayed in the Activity view to only events within that filter. For example, the Actions filter will result in only provider actions being shown in the Activity view. A provider may select the actions view to determine the latest action related to this patient and if additional action is necessary.

In some examples, the activity view 1230 may display information about events related to the patient received by the system as patient health information and maintained within the patient profile. The information about the events may be displayed in the activity view 1230 including: a type of event, a time of event, a provider associated with the event, a department associated with the event, a complaint associated with the event, and a diagnosis associated with the event. Additional information may be included with each event, selected from any information received in patient health information or maintained by the system in the patient profile. A user may select any of the events to see a view of all information associated with the event. The activity view 1230 may additionally provide a Log Action input 1231, which may be selected by a provider to record a provider action related to the patient. Logging the action may involve selecting an action type and adding notes associated with the action, as discussed with relation to FIG. 11C.

In some examples, the diagnosis 1240 view may allow users to filter a list of patient diagnoses by codes. The displayed list of patient diagnoses may include information related to the type of diagnosis, a provider associated with the diagnosis and a time of the diagnosis. Additional information may be included with each diagnosis, selected from any information received in patient health information or maintained by the system in the patient profile. A user may select any of the diagnoses to see a view of all information associated with the diagnosis.

The GUI 1100 may additionally have one or more tabs 1250 that allow a user to access different portions of the GUI and view additional information. In the example of FIGS. 11A-13, there are four tabs 1250 which a user may select from: My Patients, Performance, Community and Administration. Each tab may be displayed as a tab title and may include additional information such as a symbol. The tab currently displayed by the GUI may be displayed differently, such as in FIGS. 11A-11C, where the My Patients tab 1250A has a dark rectangle with rounded corners surrounding it.

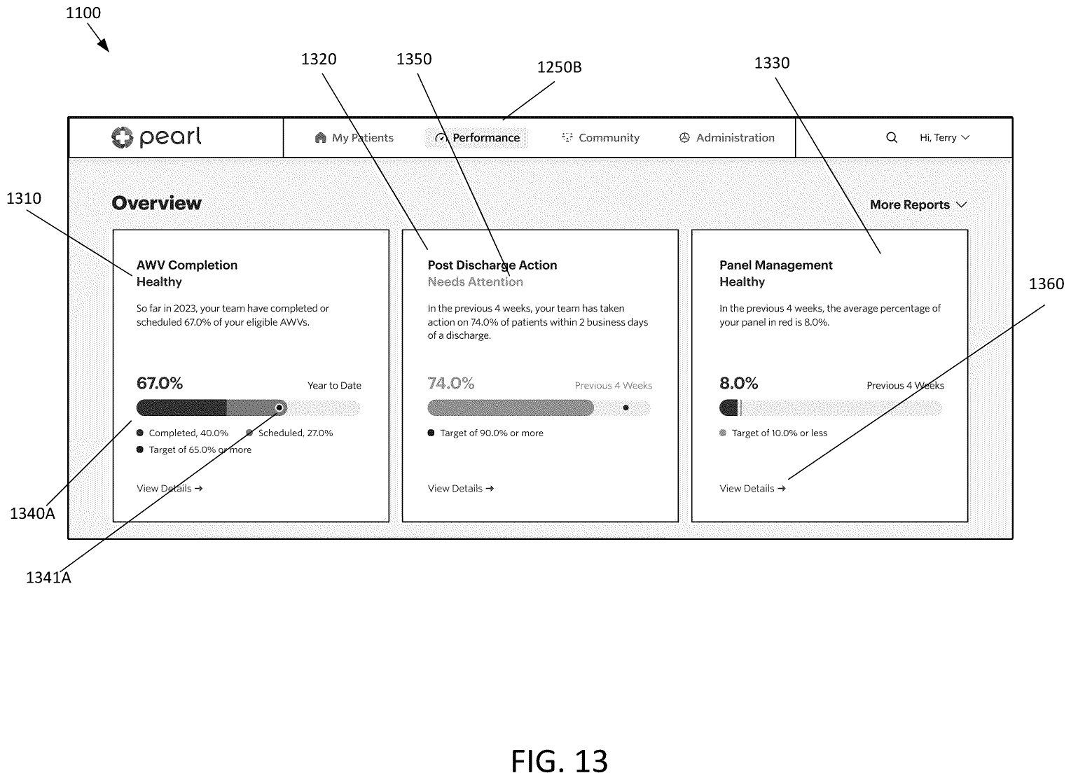

In some examples, a user may also select Performance tab 1250B at the top of GUI 1100. FIG. 13 illustrates a view of the GUI 1100 when the performance tab 1250B is selected. The Performance tab 1250B may provide a display of one or more metrics associated with the performance of the healthcare network in meeting patient needs. These metrics may be the metrics discussed with relation to FIG. 10. Each metric section may include a current value, representing the value of the metric over a representative time period. For example, the AWV section 1310 displays that 67% of patients have had AWVs completed or scheduled. Each metric section may additionally include a graph of the metric value 1340. The metric value graphs may display information including a current value, a target value and a category of the current value. For example, the current value of the AWV metric is shown by graph 1340A. The bar portion of the graph is split into two categories, completed, shown by the darker color and scheduled shown by the lighted sharded color. The target metric value may be shown as a dot 1341A in the graph. Each metric section may additionally include an alert if the metric is a threshold amount away from a target. For example, the Post Discharge Action target has a Needs Attention alert 1350 because the current value of 74% is below the target of 90%. An alert may be generated based on how far, in a negative direction, a current value is from the target value, for example the alert may be generated if the current value is 5%, 10%, 20%, or a user selected amount away from the target. Each metric section may additionally include a view details button 1360 for a user to select. Selecting the view details button will result in the user being taken to a view displaying more information related to the current metric value and the reasons for the current metric value.

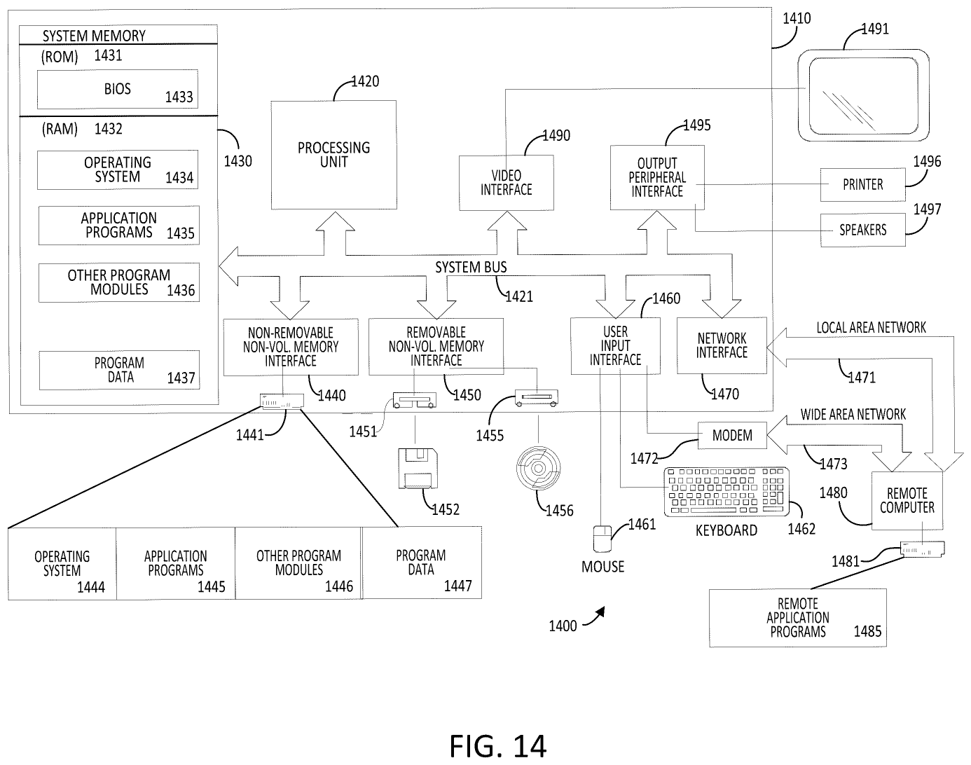

FIG. 14 shows a block diagram of an exemplary computing device, in accordance with some embodiments of the technology described herein. The computing system environment 1400 is only one example of a suitable computing environment and is not intended to suggest any limitation as to the scope of use or functionality of the technology described herein.

The technology described herein is operational with numerous other general purpose or special purpose computing system environments or configurations. Examples of well-known computing systems, environments, and/or configurations that may be suitable for use with the technology described herein include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The technology described herein may also be practiced in distributed computing environments where tasks are performed by remote processing devices that are linked through a communications network. In a distributed computing environment, program modules may be located in both local and remote computer storage media including memory storage devices.

With reference to FIG. 14, an exemplary system for implementing the technology described herein includes a general-purpose computing device in the form of a computer 1410. Components of computer 1410 may include, but are not limited to, a processing unit 1420, a system memory 1430, and a system bus 1421 that couples various system components including the system memory to the processing unit 1420. The system bus 1421 may be any of several types of bus structures, including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

Computer 1410 typically includes a variety of computer readable media. Computer readable media can be any available media that can be accessed by computer 1410 and includes both volatile and nonvolatile media, removable and non-removable media. By way of example, and not limitation, computer readable media may comprise computer storage media and communication media. Computer storage media includes volatile and nonvolatile, removable, and non-removable media implemented in any method or technology for storage of information, such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 1410. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer readable media.

The system memory 1430 includes computer storage media in the form of volatile and/or nonvolatile memory such as read only memory (ROM) 1431 and random-access memory (RAM) 1432. A basic input/output system 1433 (BIOS), containing the basic routines that help to transfer information between elements within computer 1410, such as during start-up, is typically stored in ROM 1431. RAM 1432 typically contains data and/or program modules that are immediately accessible to and/or presently being operated on by processing unit 1420. By way of example, and not limitation, FIG. 14 illustrates operating system 1434, application programs 1435, other program modules 1436, and program data 1437.

The computer 1410 may also include other removable/non-removable, volatile or nonvolatile computer storage media. By way of example only, FIG. 14 illustrates a hard disk drive 1441 that reads from or writes to non-removable, nonvolatile magnetic media, a flash drive 1451 that reads from or writes to a removable, nonvolatile memory 1452 such as flash memory, and an optical disk drive 1455 that reads from or writes to a removable, nonvolatile optical disk 1456 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 1441 is typically connected to the system bus 1421 through a non-removable memory interface such as interface 1440, and magnetic disk drive 1451 and optical disk drive 1455 are typically connected to the system bus 1421 by a removable memory interface, such as interface 1450.

The drives and their associated computer storage media described above and illustrated in FIG. 14, provide storage of computer readable instructions, data structures, program modules and other data for the computer 1410. In FIG. 14, for example, hard disk drive 1441 is illustrated as storing operating system 1444, application programs 1445, other program modules 1446, and program data 1447. Note that these components can either be the same as or different from operating system 1434, application programs 1435, other program modules 1436, and program data 1437. Operating system 1444, application programs 1445, other program modules 1446, and program data 1447 are given different numbers here to illustrate that, at a minimum, they are different copies. An actor may enter commands and information into the computer 1410 through input devices such as a keyboard 1462 and pointing device 1461, commonly referred to as a mouse, trackball, or touch pad. Other input devices (not shown) may include a microphone, joystick, game pad, satellite dish, scanner, or the like. These and other input devices are often connected to the processing unit 1420 through a user input interface 1460 that is coupled to the system bus but may be connected by other interface and bus structures, such as a parallel port, game port or a universal serial bus (USB). A monitor 1491 or other type of display device is also connected to the system bus 1421 via an interface, such as a video interface 1490. In addition to the monitor, computers may also include other peripheral output devices such as speakers 1497 and printer 1496, which may be connected through an output peripheral interface 1495.

The computer 1410 may operate in a networked environment using logical connections to one or more remote computers, such as a remote computer 1480. The remote computer 1480 may be a personal computer, a server, a router, a network PC, a peer device, or other common network node, and typically includes many or all of the elements described above relative to the computer 1410, although only a memory storage device 1481 has been illustrated in FIG. 14. The logical connections depicted in FIG. 14 include a local area network (LAN) 1471 and a wide area network (WAN) 1473 but may also include other networks. Such networking environments are commonplace in offices, enterprise-wide computer networks, intranets, and the Internet.

When used in a LAN networking environment, the computer 1410 is connected to the LAN 1471 through a network interface or adapter 1470. When used in a WAN networking environment, the computer 1410 typically includes a modem 1472 or other means for establishing communications over the WAN 1473, such as the Internet. The modem 1472, which may be internal or external, may be connected to the system bus 1421 via the actor input interface 1460, or other appropriate mechanism. In a networked environment, program modules depicted relative to the computer 1410, or portions thereof, may be stored in the remote memory storage device. By way of example, and not limitation, FIG. 14 illustrates remote application programs 1485 as residing on memory device 1481. It will be appreciated that the network connections shown are exemplary and other means of establishing a communications link between the computers may be used.

Having thus described several aspects of at least one embodiment of the technology described herein, it is to be appreciated that various alterations, modifications, and improvements will readily occur to those skilled in the art. Such alterations, modifications, and improvements are intended to be part of this disclosure and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Further, it should be appreciated that a computer may be embodied in any of a number of forms, such as a rack-mounted computer, a desktop computer, a laptop computer, a tablet computer, a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy disks, compact disks (CD), optical disks, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. A computer-readable storage medium includes any computer memory configured to store software, for example, the memory of any computing device such as a smart phone, a laptop, a desktop, a rack-mounted computer, or a server (e.g., a server storing software distributed by downloading over a network, such as an app store). As used herein, the term “computer-readable storage medium” encompasses only a non-transitory computer-readable medium that can be considered to be a manufacture (i.e., article of manufacture) or a machine. Alternatively, or additionally, aspects of the technology described herein may be embodied as a computer readable medium other than a computer-readable storage medium, such as a propagating signal.

The terms “program” or “software” are used herein in a generic sense to refer to any type of computer code or set of processor-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor but may be distributed in a modular fashion among a number of different computers or processors to implement various aspects of the technology described herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which examples are provided herein including with reference to FIGS. 8A-D and 9. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

Although it is appreciated that the system may provide recommendations to physicians, various aspects of this system may be integrated into one or more expert systems that are capable of rendering medical advice.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately” and “about” may be used to mean within +20% of a target value in some embodiments, within +10% of a target value in some embodiments, within +5% of a target value in some embodiments, within +2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Various aspects are described in this disclosure, which include, but are not limited to, the following aspects:

1. A method for facilitating medical care of patients, the method comprising: using one or more processors to perform: obtaining one or more electronic patient health datasets related to a plurality of patients; generating a plurality of patient profiles from the one or more electronic patient health datasets, wherein each patient profile is stored using at least one data structure, and wherein a first patient profile of the plurality of patient profiles is generated by: analyzing data from the one or more electronic patient health datasets to identify electronic patient health data associated with a respective first patient; analyzing electronic patient health data associated with the first patient to determine electronic patient health data associated with one or more patient features of the first patient; determining a severity score for the first patient profile using electronic patient health data associated with the one or more patient features; analyzing the electronic patient health data associated with the first patient to identify electronic patient health data associated with one or more medical events of the first patient; determining an urgency score for the first patient profile using the electronic patient health data associated with the one or more medical events; and storing the severity and urgency score determined for the first patient profile in at least one data structure associated with the first patient profile; and displaying ordered representations of profiles of the plurality of patient profiles, wherein the representations are ordered based at least in part on the severity score of the respective profile.

2. The method of aspect 1 or any other preceding aspect, wherein the one or more patient features comprise at least one of: an age of the first patient, a gender of the first patient, and chronic conditions of the first patient.

3. The method of aspect 2 or any other preceding aspect, wherein the patient features further comprise at least one of: an income associated with the first patient, a location associated with the first patient, an education level associated with the first patient, an employment status associated with the first patient, a family condition associated with the first patient, one or more behaviors associated with the first patient, a measure of health care access associated with the first patient, acute conditions of the first patient, and comorbidities associated with the first patient.

4. The method of aspect 1 or any other preceding aspect, wherein the one or more medical events include events selected from: medical facility admission, medical facility discharge, medical facility transfer, and medical provider actions.

5. The method of aspect 4 or any other preceding aspect, further comprising determining, from the electronic patient health data associated with the one or more medical events, signals related to the first patient, wherein each signal determined from at least one event.

6. The method of aspect 5 or any other preceding aspect, wherein at least one signal of the signals is determined using a trained machine learning model.

7. The method of aspect 5 or any other preceding aspect, wherein the signals include signals selected from: time since admission to a medical facility, time since discharge from a medical facility, time until a recommended follow-up, time since a recommended follow-up, and time since last annual wellness visit.

8. The method of aspect 7 or any other preceding aspect, wherein the signals further include signals selected from: a measure of prescription adherence, a probability of readmission, a measure of a need for advanced care planning and a measure of a need for specialist referral.

9. The method of aspect 7 or any other preceding aspect, further comprising, for the first patient profile, determining a suggested action plan for consideration, the action plan comprising a plurality of actions and being based at least on the signals associated with the first patient.

10. The method of aspect 9 or any other preceding aspect, wherein determining the action plan comprises analyzing each of the signals associated with the first patient and adding at least one action associated with each signal to the action plan.

11. The method of aspect 10 or any other preceding aspect, wherein determining the action plan comprises analyzing each of the signals associated with the first patient and adding at least one action associated with two or more signals to the action plan.

12. The method of aspect 10 or any other preceding aspect, wherein the suggested action plan comprises one or more suggested actions for consideration selected from: contact the first patient via phone, contact the first patient in medical facility, schedule an appointment for the first patient, schedule an annual wellness visit for the first patient, refer the first patient to a specialist, and recommend a medical intervention for the first patient.

13. The method of aspect 7 or any other preceding aspect, wherein the urgency score of the first patient profile increases in response to the time until a recommended follow-up signal crossing a threshold amount of time.

14. The method of aspect 7 or any other preceding aspect, wherein the urgency score of the first patient profile increases in response to an increase in the time since admission signal, an increase in the time since discharge signal, and an increase in the time since transfer signal, until a signal indicating a recommended follow-up has occurred is received.

15. The method of aspect 7 or any other preceding aspect, further comprising determining one or more performance metrics based at least in part on changes in urgency scores associated with patient profiles, changes in value of the time until a recommended follow-up signal, changes in value of the time since a recommended follow-up signal, and changes in value of the time since last annual wellness visit signal.

16. The method of aspect 1 or any other preceding aspect, wherein an increase in the urgency score of the first patient profile indicates the first patient has a greater predicted need for proactive attention from medical providers.

17. The method of aspect 1 or any other preceding aspect, wherein an increase in the severity score of the first patient profile indicates the first patient has experienced a decrease in overall health.

18. The method of aspect 7 or any other preceding aspect, further comprising receiving data related to one or more events related to the first patient; and in response to receiving data related to one or more events related to the first patient, updating the respective urgency score for the first patient profile.

19. A system for facilitating medical care of patients, the system comprising: at least one computer hardware processor; a display; and at least one non-transitory computer-readable storage medium storing processor-executable instructions that, when executed by the at least one computer hardware processor, cause the at least one computer hardware processor to perform a method, the method comprising: obtaining one or more electronic patient health datasets related to a plurality of patients; generating a plurality of patient profiles from the one or more electronic patient health datasets, wherein each patient profile is stored using at least one data structure, and wherein a first patient profile of the plurality of patient profiles is generated by: analyzing data from the one or more electronic patient health datasets to identify electronic patient health data associated with a respective first patient; analyzing electronic patient health data associated with the first patient to determine electronic patient health data associated with one or more patient features of the first patient; determining a severity score for the first patient profile using electronic patient health data associated with the one or more patient features; analyzing the electronic patient health data associated with the first patient to identify electronic patient health data associated with one or more medical events of the first patient; determining an urgency score for the first patient profile using the electronic patient health data associated with the one or more medical events; and storing the severity and urgency score determined for the first patient profile in at least one data structure associated with the first patient profile; and displaying, on the display, ordered representations of profiles of the plurality of patient profiles, wherein the representations are ordered based at least in part on the severity score of the respective profile.

20. At least one non-transitory computer-readable storage medium storing processor-executable instructions that, when executed by at least one computer hardware processor, cause the at least one computer hardware processor to perform a method, the method comprising: obtaining one or more electronic patient health datasets related to a plurality of patients; generating a plurality of patient profiles from the one or more electronic patient health datasets, wherein each patient profile is stored using at least one data structure, and wherein a first patient profile of the plurality of patient profiles is generated by: analyzing data from the one or more electronic patient health datasets to identify electronic patient health data associated with a respective first patient; analyzing electronic patient health data associated with the first patient to determine electronic patient health data associated with one or more patient features of the first patient; determining a severity score for the first patient profile using electronic patient health data associated with the one or more patient features; analyzing the electronic patient health data associated with the first patient to identify electronic patient health data associated with one or more medical events of the first patient; determining an urgency score for the first patient profile using the electronic patient health data associated with the one or more medical events; and storing the severity and urgency score determined for the first patient profile in at least one data structure associated with the first patient profile; and displaying ordered representations of profiles of the plurality of patient profiles, wherein the representations are ordered based at least in part on the severity score of the respective profile.

21. A method for facilitating medical care of patients, the method comprising: obtaining one or more electronic patient health datasets related to a plurality of patients; processing the one or more electronic patient health datasets to generate a plurality of patient profiles, each patient profile of the plurality or patient profiles being associated with a respective patient of the plurality of patients; and displaying, on a device screen, a patient map, wherein the patient map includes graphical representations of one or more patient profiles of the plurality of patient profiles, and wherein the patient map includes, for each graphical representation, an indication of predicted urgency for attention for the patient associated with the graphical representation.

22. The method according to aspect 21 or any other preceding aspect, wherein the act of displaying the patient map comprises displaying the graphical representations in an order on the device screen responsive to a last touchpoint with each of the respective patients.

23. The method according to aspect 21 or any other preceding aspect, wherein the patient map is a rectangular map having a vertical axis and a horizontal axis, and wherein the graphical representations are ordered in a direction parallel to the vertical axis based on a severity score of the respective patient profile and the graphical representations are ordered in a direction parallel to the horizontal axis based on a signal indicative of days since last touchpoint of the respective patient profile.

24. The method of aspect 21 or any other preceding aspect, further comprising displaying on the device screen a patient list, wherein the patient list includes a graphical list representation of one or more patient profiles of the plurality of patient profiles.

25. The method of aspect 24 or any other preceding aspect, wherein the graphical list representations are ordered based on an urgency score of each respective patient profile.

26. The method of aspect 24 or any other preceding aspect, further comprising displaying one or more patient map filters on the device screen.

27. The method of aspect 26 or any other preceding aspect, wherein the one or more patient map filters include a provider filter and a reason for urgency filter.

28. The method of aspect 27 or any other preceding aspect, further comprising in response to receiving a user input to the provider filter, displaying a graphical representation of each respective patient profile of a subset of patient profiles of the plurality of patient profiles, wherein each patient profile of the subset of patient profiles is associated with a provider input by the user to the provider filter.

29. The method of aspect 24 or any other preceding aspect, wherein the graphical representation of each patient profile of the plurality of patient profiles has a color, the color being associated with an urgency score of the respective patient profile.

30. The method of aspect 24 or any other preceding aspect, wherein the graphical representation of each patient profile of the plurality of patient profiles has a size, the size being associated with an urgency score of the respective patient profile.

31. The method of aspect 4 or any other preceding aspect, wherein the graphical representation of each patient profile of the plurality of patient profiles has a shape, the shape being associated with an urgency score of the respective patient profile.

32. The method of aspect 24 or any other preceding aspect, further comprising, in response to receiving a user selection of a particular graphical representation of a patient profile of the patient map, displaying a patient summary comprising patient demographic information, patient diagnoses, and patient events.

33. The method of aspect 24 or any other preceding aspect, further comprising, in response to receiving a user selection of a particular graphical representation of a patient profile of the patient map, displaying a detailed patient view on the device screen.

34. The method of aspect 33 or any other preceding aspect, wherein the detailed patient view comprises patient demographic information, patient activity information, patient diagnosis information, a patient summary, and patient medication information.

35. The method of aspect 34 or any other preceding aspect, wherein the detailed patient view comprises a log action input.

36. The method of aspect 35 or any other preceding aspect, further comprising in response to a user input to the log action input, displaying an action menu associated with the respective patient profile.

37. A system for facilitating medical care of patients, the system comprising: at least one computer hardware processor; a display; and at least one non-transitory computer-readable storage medium storing processor-executable instructions that, when executed by the at least one computer hardware processor, cause the at least one computer hardware processor to perform a method, the method comprising: obtaining one or more electronic patient health datasets related to a plurality of patients; processing the one or more electronic patient health datasets to generate a plurality of patient profiles, each patient profile of the plurality or patient profiles associated with a respective patient of the plurality of patients; and displaying, on the display, a patient map, wherein the patient map includes graphical representations of one or more patient profiles of the plurality of patient profiles, and wherein the patient map includes, for each graphical representation, an indication of predicted urgency for attention for the patient associated with the graphical representation.

38. The system of aspect 37, wherein the act of displaying the patient map comprises displaying the graphical representations in an order on the display responsive to a last touchpoint with each of the respective patients.

39. The system of aspect 37, wherein the patient map is a rectangular map having a vertical axis and a horizontal axis, and wherein the graphical representations are ordered in a direction parallel to the vertical axis based on a severity score of the respective patient profile and the graphical representations are ordered in a direction parallel to the horizontal axis based on a signal indicative of days since last touchpoint of the respective patient profile.

40. At least one non-transitory computer-readable storage medium storing processor-executable instructions that, when executed by at least one computer hardware processor, cause the at least one computer hardware processor to perform a method, the method comprising: obtaining one or more electronic patient health datasets related to a plurality of patients; processing the one or more electronic patient health datasets to generate a plurality of patient profiles, each patient profile of the plurality or patient profiles associated with a respective patient of the plurality of patients; and displaying, on a device screen, a patient map, wherein the patient map includes graphical representations of one or more patient profiles of the plurality of patient profiles, and wherein the patient map includes, an indication of predicted urgency for attention for the patient associated with the graphical representation.

What is claimed is:

1. A method for facilitating medical care of patients, the method comprising:
using one or more processors to perform:
obtaining one or more electronic patient health datasets related to a plurality of patients;
generating a plurality of patient profiles from the one or more electronic patient health datasets, wherein each patient profile is stored using at least one data structure, and wherein a first patient profile of the plurality of patient profiles is generated by:
analyzing data from the one or more electronic patient health datasets to identify electronic patient health data associated with a first patient;
analyzing electronic patient health data associated with the first patient to determine electronic patient health data associated with one or more patient features of the first patient;
determining a severity score for the first patient profile using electronic patient health data associated with the one or more patient features;
analyzing the electronic patient health data associated with the first patient to identify electronic patient health data associated with one or more medical events of the first patient;
determining an urgency score for the first patient profile using the electronic patient health data associated with the one or more medical events; and
storing the severity and urgency score determined for the first patient profile in at least one data structure associated with the first patient profile; and
displaying ordered representations of profiles of the plurality of patient profiles, wherein the representations are ordered based at least in part on the severity score of the respective profile.

2. The method of claim 1, wherein the one or more patient features comprise at least one of: an age of the first patient, a gender of the first patient, and chronic conditions of the first patient.

3. The method of claim 2, wherein the patient features further comprise at least one of: an income associated with the first patient, a location associated with the first patient, an education level associated with the first patient, an employment status associated with the first patient, a family condition associated with the first patient, one or more behaviors associated with the first patient, a measure of health care access associated with the first patient, acute conditions of the first patient, and comorbidities associated with the first patient.

4. The method of claim 1, wherein the one or more medical events include events selected from: medical facility admission, medical facility discharge, medical facility transfer, and medical provider actions.

5. The method of claim 4, further comprising determining, from the electronic patient health data associated with the one or more medical events, signals related to the first patient, wherein each signal determined from at least one event.

6. The method of claim 5, wherein at least one signal of the signals is determined using a trained machine learning model.

7. The method of claim 5, wherein the signals include signals selected from: time since admission to a medical facility, time since discharge from a medical facility, time until a recommended follow-up, time since a recommended follow-up, and time since last annual wellness visit.

8. The method of claim 7, wherein the signals further include signals selected from: a measure of prescription adherence, a probability of readmission, a measure of a need for advanced care planning and a measure of a need for specialist referral.

9. The method of claim 7, further comprising, for the first patient profile, determining a suggested action plan, the suggested action plan comprising a plurality of actions and being based at least on the signals associated with the first patient.

10. The method of claim 9, wherein determining the suggested action plan comprises analyzing each of the signals associated with the first patient and adding at least one action associated with each signal to the suggested action plan.

11. The method of claim 10, wherein determining the suggested action plan comprises analyzing each of the signals associated with the first patient and adding at least one action associated with two or more signals to the suggested action plan.

12. The method of claim 10, wherein the suggested action plan comprises one or more suggested actions for consideration, selected from: contact the first patient via phone, contact the first patient in medical facility, schedule an appointment for the first patient, schedule an annual wellness visit for the first patient, refer the first patient to a specialist, and recommend a medical intervention for the first patient.

13. The method of claim 7, wherein the urgency score of the first patient profile increases in response to the time until a recommended follow-up signal crossing a threshold amount of time.

14. The method of claim 7, wherein the urgency score of the first patient profile increases in response to an increase in the time since admission signal, an increase in the time since discharge signal, and an increase in the time since transfer signal, until a signal indicating a recommended follow-up has occurred is received.

15. The method of claim 7, further comprising determining one or more performance metrics based at least in part on changes in urgency scores associated with patient profiles, changes in value of the time until a recommended follow-up signal, changes in value of the time since a recommended follow-up signal, and changes in value of the time since last annual wellness visit signal.

16. The method of claim 1, wherein an increase in the urgency score of the first patient profile indicates the first patient has a greater predicted need for proactive attention from medical providers.

17. The method of claim 1, wherein an increase in the severity score of the first patient profile indicates the first patient has experienced a decrease in overall health.

18. The method of claim 7, further comprising receiving data related to one or more events related to the first patient; and in response to receiving data related to one or more events related to the first patient, updating the respective urgency score for the first patient profile.

19. A system for facilitating medical care of patients, the system comprising:
at least one computer hardware processor;
a display; and
at least one non-transitory computer-readable storage medium storing processor-executable instructions that, when executed by the at least one computer hardware processor, cause the at least one computer hardware processor to perform a method, the method comprising:
obtaining one or more electronic patient health datasets related to a plurality of patients;
generating a plurality of patient profiles from the one or more electronic patient health datasets, wherein each patient profile is stored using at least one data structure, and wherein a first patient profile of the plurality of patient profiles is generated by:
analyzing data from the one or more electronic patient health datasets to identify electronic patient health data associated with a first patient;
analyzing electronic patient health data associated with the first patient to determine electronic patient health data associated with one or more patient features of the first patient;
determining a severity score for the first patient profile using electronic patient health data associated with the one or more patient features;
analyzing the electronic patient health data associated with the first patient to identify electronic patient health data associated with one or more medical events of the first patient;
determining an urgency score for the first patient profile using the electronic patient health data associated with the one or more medical events; and
storing the severity and urgency score determined for the first patient profile in at least one data structure associated with the first patient profile; and
displaying, on the display, ordered representations of profiles of the plurality of patient profiles, wherein the representations are ordered based at least in part on the severity score of the respective profile.

20. At least one non-transitory computer-readable storage medium storing processor-executable instructions that, when executed by at least one computer hardware processor, cause the at least one computer hardware processor to perform a method, the method comprising:
obtaining one or more electronic patient health datasets related to a plurality of patients;
generating a plurality of patient profiles from the one or more electronic patient health datasets, wherein each patient profile is stored using at least one data structure, and wherein a first patient profile of the plurality of patient profiles is generated by:
analyzing data from the one or more electronic patient health datasets to identify electronic patient health data associated with a first patient;
analyzing electronic patient health data associated with the first patient to determine electronic patient health data associated with one or more patient features of the first patient;
determining a severity score for the first patient profile using electronic patient health data associated with the one or more patient features;
analyzing the electronic patient health data associated with the first patient to identify electronic patient health data associated with one or more medical events of the first patient;
determining an urgency score for the first patient profile using the electronic patient health data associated with the one or more medical events; and
storing the severity and urgency score determined for the first patient profile in at least one data structure associated with the first patient profile; and
displaying ordered representations of profiles of the plurality of patient profiles, wherein the representations are ordered based at least in part on the severity score of the respective profile.
